(19) United States
(12) Patent Application Publication
(10) Pub. No.: US 2025/0255559 A1
(43) Pub. Date: Aug. 14, 2025

(54) System And Method for Remote Passive Monitoring of Visual Health-Related Indicators

(71) Applicant: Celloscope Ltd., Tel Aviv (IL)

(72) Inventors: Yuval NAVEH, Haifa (IL); Tamir ALOUSH, Kiryat Ono (IL); Levi TEITZ, Tel Aviv (IL); Shahar DAVIDSON, Rehovot (IL); Tomer SHUSSMAN, Kfar Saba (IL)

(21) Appl. No.: 19/056,378

(22) Filed: Feb. 18, 2025

Related U.S. Application Data
(63) Continuation-in-part of application No. 18/939,288, filed on Nov. 6, 2024.
(63) Continuation-in-part of application No. 18/975,890, filed on Dec. 10, 2024.
(63) Continuation-in-part of application No. 19/049,400, filed on Feb. 10, 2025.
(63) Continuation-in-part of application No. 19/051,712, filed on Feb. 12, 2025.
(60) Provisional application No. 63/596,479, filed on Nov. 6, 2023.
(60) Provisional application No. 63/612,587, filed on Dec. 20, 2023.
(60) Provisional application No. 63/557,740, filed on Feb. 26, 2024.
(60) Provisional application No. 63/557,747, filed on Feb. 26, 2024.
(60) Provisional application No. 63/557,753, filed on Feb. 26, 2024.
(60) Provisional application No. 63/557,757, filed on Feb. 26, 2024.
(60) Provisional application No. 63/557,762, filed on Feb. 26, 2024.

(51) Int. Cl. A61B 5/00; A61B 3/10; A61B 5/11

(52) U.S. Cl. CPC A61B 5/7282; A61B 3/102; A61B 5/112

(57) ABSTRACT

An improved health-care system comprising an output indication generating functionality which may be configured to screen for visual health deterioration e.g. by generating an output indication aka alert to at least one entity to alert for visual health deterioration; and/or visual health monitoring functionality including a hardware processor which may be configured to monitor persons carrying cellphones for at least one gait impairment indicator which may be characteristic of deterioration of visual health, e.g. by performing gait analysis, at intervals, typically on data generated by the persons' cellphones' IMUs, and, typically to, accordingly, command the output indication generating functionality to generate the output indication e.g. when at least one gait impairment indicator monitored complies with at least one alert criterion.

### CROSS-REFERENCE TO RELATED APPLICATIONS

The present application is a continuation-in-part of U.S. application Ser. No. 18/939,288, filed Nov. 6, 2024, and of U.S. application Ser. No. 18/975,890, filed Dec. 10, 2024, and of U.S. application Ser. No. 19/049,400, filed Feb. 10, 2025, and of U.S. application Ser. No. 19/051,712 entitled “System, Method, and Computer Program Product for Tracking Cognitive Status Based On Repeated Mobility Assessments” filed Feb. 12, 2025, the entire contents of each of which being hereby fully incorporated herein by reference. The present application further claims benefit, directly or indirectly, of the following provisional applications, the entire contents of each of which being fully incorporated herein by reference: Application No. 63/557,740, filed Feb. 26, 2024 and entitled “Mobility Assessment in Visually Impaired Individuals”; Application No. 63/557,747, filed Feb. 26, 2024; Application No. 63/557,753, filed Feb. 26, 2024; Application No. 63/557,757, filed Feb. 26, 2024, Application No. 63/557,762, filed Feb. 26, 2024; Application No. 63/596,479, filed Nov. 6, 2023; and Application No. 63/612,587, filed Dec. 20, 2023.

### FIELD OF THIS DISCLOSURE

The present invention relates generally to computerized analysis of motion, and more particularly to computerized analysis of human motion which receives sensor outputs borne by a human, typically in real time.

### BACKGROUND FOR THIS DISCLOSURE

OneStep is an FDA-listed medical app, downloadable from GooglePlay, that uses smartphone motion sensors to provide immediate, clinically-validated feedback on gait inter alia.

The disclosures of all publications and patent documents mentioned above and elsewhere in the specification, and of the publications and patent documents cited therein directly or indirectly, are hereby incorporated herein by reference in their entirety. If the incorporated material is inconsistent with the express disclosure herein, the interpretation is that the express disclosure herein describes certain embodiments, whereas the incorporated material describes other embodiments. Definition/s within the incorporated material may be regarded as one possible definition for the term/s in question.

### SUMMARY OF CERTAIN EMBODIMENTS

Certain embodiments of the present invention seek to provide circuitry typically comprising at least one processor in communication with at least one memory, with instructions stored in such memory executed by the processor to provide functionalities which are described herein in detail. Any functionality described herein may be firmware-implemented or processor-implemented, as appropriate.

Certain embodiments seek to provide non-clinic monitoring of visual health and/or visual abilities and use of monitoring outputs for screening and/or for monitoring disease progression and/or for treatment efficacy monitoring; it is appreciated that certain visual health parameters are indicators of various types of general disease including hypertension, brain tumors and more.

Certain embodiments seek to provide remote, typically passive, screening for new (or worsening) and potentially serious and urgent eye conditions, e.g. by correlating standard visual impairment outcome measures with standard mobility measures; the measures may be derived by analyzing IMU (inertial measurement unit) data during passive gait monitoring. The system may, for example, correlate IOP (intraocular pressure) or changes therein, with standard mobility measures or changes therein.

Alternatively, or in addition, the system may detect shuffling and/or head position changes, e.g. staring at the ground or relevant detectable heuristics generally by analyzing IMU data during passive gait monitoring.

Certain embodiments seek to augment or at least partly replace or improve routine periodic in-clinic follow-up of patients' aka end users' visual health with continuous (e.g. once per hour or more often, or once per day or once per week or once per month or once per year, or less often) monitoring of visual health based on gait analysis of end users' IMU data generated by the end-user's cellphones' IMUs. Visual health monitoring may include repeatedly, e.g. periodically, generating an estimate of at least one standard visual health outcome such as IOP, from gait parameters produced by the gait analysis.

When the standard outcome measures suggest visual deterioration, the system may alert the end-user's HMO (Health Maintenance Organization) (and/or end-user himself) and/or may generate such alerts for persons for whom heuristics such as shuffling or staring at the floor (references herein to “floor” and “ground” may be interchanged) have been detected, and the HMO may, responsively, summon these end-users for an in-clinic eye doctor appointment. Optionally, the system may receive diagnoses made during or subsequent to such appointments (e.g. “glaucoma” or “no pathology found”) and, using these diagnoses as training data, may eventually propose diagnoses which may or may not be confirmed by a human domain expert.

Certain embodiments seek to provide a system for mobility assessment in visually impaired individuals.

Certain embodiments include a mobile device with IMU and/or a mobile app and/or a caregiver dashboard, and/or a hardware processor configured to receive data and communicate with the mobile app and the caregiver dashboard. Typically, the processor continuously (e.g. more frequently than traditional once-or-twice-yearly in-clinic visits) assesses the mobile app user's functional status and enables the caregiver to act in different care actions based on at least one mobility measure.

Certain embodiments include caregiver's tools that depend on the mobility assessment of the individual and the plural patients. Therefore, the capability of motion analysis, including gait and mobility measurement using a mobile app on a single device with IMU, is considered.

At least the following embodiments are thus provided:

Embodiment 1. An improved health-care system comprising:
an output indication generating functionality configured to screen for visual health deterioration by generating an output indication aka alert to at least one entity to alert for visual health deterioration; and a visual health monitoring functionality including a hardware processor configured to monitor persons carrying cellphones for at least one gait impairment indicator which may be characteristic of deterioration of visual health, by performing gait analysis, at intervals, on data generated by the persons' cellphones' IMUs, and, accordingly, to command the output indication generating functionality to generate the output indication e.g. when at least one gait impairment indicator monitored complies with at least one alert criterion.

The alert may for example comprise an automatic email to an email address, stored in the system, of an entity e.g. HMO or an automatic SMS to a cellphone number, stored in the system, of the entity where the email or SMS or other alert may for example comprise a report document describing a given user's deterioration (e.g. describing a gait parameter deterioration and/or visual impairment which may be characteristic of deterioration of visual health). Alternatively or in addition, the alert e.g. report document may be consumed via web dashboard or via API with, say, an EMR software platform. The EMR may have appointment scheduling functionality such as Epic Systems or Cerner so as to automatically schedule an appointment responsive to certain defined deterioration e.g. responsive to each alert triggered by gait impairment indicator/s such as newly decreased gait parameter values, new presence of shuffling, standard outcomes that have newly departed from normal range. Or, appointment scheduling may follow a priority order defined over the HMO's patients aka end-users and an alert regarding patient P may increase patient P's priority ranking from low to high, say.

Embodiment 2: The system of any embodiment herein, wherein the alert stipulates at least one change in gait parameter/s relative to value/s of the gait parameter/s that has/have characterized an individual user in the past.

The extent and/or type of change which should trigger an output indication may be defined by the alert criterion. For example, the alert criterion may stipulate that the output indication should be generated if one (say) or more gait parameters such as, say, stride length, cadence or gait speed (or a certain logical combination thereof) drop by at least (say) 10% or 20% over a time period of (say) a year or less. Typically values for the gait parameters are computed for this purpose initially per stride (using any suitable method for partitioning or segmenting a passive walk which is (say) 1 minute long into strides (e.g. as described in co-owned U.S. Ser. No. 16/659,832, published as US 2020/0289027, entitled “Assessment Of A User's Gait” and dated 22 Oct. 2019 which is hereby incorporated herein by reference in its entirety). These values are then typically averaged over all strides taken during a passive walk which may be, say, a minute long and may include, say, several dozen strides assuming a given stride is about 0.5-2 sec in duration (or a weekly average may be computed for each gait parameter e.g. by averaging over all passive walks measured during a given week). Passive walks are typically measured repeatedly for each user, e.g. daily. Alternatively or in addition, the alert criterion may stipulate that if a user's standard deviation (over all strides in a passive walk e.g.) grows by, say, 20% or more relative to her or his standard deviation (over all strides in a passive walk e.g.) a year or less before, this should trigger an output indication. Again weekly values of such standard deviations may be compared to one another. Alternatively or in addition, the alert criterion may stipulate that if a user begins to shuffle or to trip more frequently, this should trigger an output indication. Alternatively or in addition, the alert criterion may stipulate that new departures of estimated standard outcomes from population norms for these outcomes, should trigger an output indication. The above are merely exemplary of the particular scheme that may be employed to compare passive walk/s from time-range or date range x to passive walk/s from time- or date-range y, thereby to monitor and/or track a user's gait parameters characterizing these passive walks, over time, e.g. to identify visual deterioration or for other purposes.

Embodiment 3. The system of any embodiment herein, wherein the alert stipulates at least one estimated standard outcome which deviates from at least one norm, stored in the system, for the standard outcome. The norm may for example comprise a range of normal values known in the medical arts for a given standard outcome (such as, say, 10-21 mmHg for IOP or 180-200 degrees for horizontal field of vision or 130-135 degrees for vertical field of vision or mesopic visual acuity of 20/30 to 20/40) e.g. for a given population or sub-population.

Embodiment 4. The system of any embodiment herein, wherein the visual impairment characteristic of deterioration of visual health comprises plural changes in plural gait parameters respectively which are machine-learned to be indicative of deterioration of visual health.

Embodiment 5. The system of any embodiment herein, wherein the estimated standard outcome, is generated using a model trained on training data including IMU data recorded for end-users labelled to indicate standard outcome results for each end-user which were generated in-clinic.

Embodiment 6. The system of any embodiment herein, wherein responsive to the alert, the system may schedule an in-clinic session with a digital retinal camera.

Embodiment 7. The system of any embodiment herein, wherein responsive to the alert, the system may schedule an in-clinic optical coherence tomography (OCT) session.

Embodiment 8. The system of any embodiment herein, wherein the estimated standard outcome, is generated using a classifier trained on training data including first IMU data recorded for first end-users labelled to indicate that, for these end-users, a given standard outcome has deteriorated, and second IMU data recorded for second end-users which is labelled to indicate that, for these end-users, the given standard outcome has not deteriorated.

Embodiment 9. The system of any embodiment herein, wherein the gait impairment characteristic of deterioration of visual health comprises an indication of shuffling generated by gait analysis.

Embodiment 10. The system of any embodiment herein, wherein the gait impairment characteristic of deterioration of visual health comprises an indication generated by gait analysis that an end-user is staring at the ground when walking.

Embodiment 11. The system of any embodiment herein, and also comprising appointment scheduling functionality configured to prompt a user and/or his eye physician, responsive to the output indication, to schedule an appointment in-clinic to undergo eye exams e.g. all or any subset of visual acuity tests: tonometry and dilated eye exams to identify at least one of macular degeneration, diabetic retinopathy, or retinal detachment.

Embodiment 12. The system of any embodiment herein, wherein upon detection of gait impairment characteristic of deterioration of visual health, the system is configured to prompt undergo remote monitoring e.g. apps e.g. for Amsler grid and/or initiate home testing e.g. to undergo a home tonometer test to measure intraocular pressure from home.

Embodiment 13. The system of any embodiment herein, wherein the at least one visual health deterioration indicator comprises an indication that a person has begun shuffling, and wherein the hardware processor comprises a classifier configured to differentiate persons who shuffle from persons who do not shuffle.

According to certain embodiments, a gait analysis system serving at least one HMO and receiving IMU data from end-users of the HMO is configured to alert (e.g. to facilitate automatic generation of an in-clinic appointment) if an end-user begins shuffling although s/he did not shuffle at all previously, or if an end-user shuffles more than s/he used to e.g. as evidenced by an end-user's stride length getting shorter and/or each stride becoming longer in duration and/or reduced toe clearance and/or differences in ground reaction force (GRF) and/or decreased knee or hip flexion, relative to the end-user's norms and/or relevant population norms, as may be stored in the system. The system may then, responsively, schedule a care action for each or at least one such end-user e.g. to determine whether shuffling is due to an onset of (or worsening of) vision impairment, Parkinson's, Multiple Sclerosis (MS), antidepressant or antipsychotic drug side effects, depression-related “psychomotor retardation”, and so forth.

Embodiment 14. The system of any embodiment herein, wherein the at least one visual health deterioration indicator comprises an indication that a person has begun staring at the ground during gait and wherein the hardware processor comprises a classifier configured to differentiate persons who stare at the ground during gait from persons who do not.

According to certain embodiments, any methods described in co-owned patent documents US 2022/0111257, US20200289027A1, both incorporated herein by reference in their entirety, may be used to determine time intervals in which a user is walking or running vs time intervals in which a user is stationary, and a person who is found to be staring at the ground during gait is monitored using gait analysis also when stationary, to prevent false alarms in which persons who stare at the ground even when stationary are reported as persons with visual deterioration.

Embodiment 15. An improved health-care method comprising:

Using an output indication generating functionality to screen for visual health deterioration by generating an output indication aka alert to at least one entity to alert for visual health deterioration; and

Using a hardware processor to monitor persons carrying cellphones for gait impairment characteristic of deterioration of visual health by repeatedly performing gait analysis on data generated by the persons' cellphones' IMUs, and, accordingly, commanding the output indication generating functionality to generate the output indication.

Embodiment 16. A computer program product, comprising a non-transitory tangible computer readable medium having computer readable program code embodied therein, the computer readable program code adapted to be executed to implement an improved health-care method comprising:

Using an output indication generating functionality to screen for visual health deterioration by generating an output indication aka alert to at least one entity to alert for visual health deterioration; and

Using a hardware processor to monitor persons carrying cellphones for gait impairment characteristic of deterioration of visual health by repeatedly performing gait analysis on data generated by the persons' cellphones' IMUs, and, accordingly, commanding the output indication generating functionality to generate the output indication.

Also provided, excluding signals, is a computer program comprising computer program code means for performing any of the methods shown and described herein when the program is run on at least one computer; and a computer program product, comprising a typically non-transitory computer-usable or -readable medium e.g. non-transitory computer-usable or -readable storage medium, typically tangible, having a computer readable program code embodied therein, the computer readable program code adapted to be executed to implement any or all of the methods shown and described herein. The operations in accordance with the teachings herein may be performed by at least one computer specially constructed for the desired purposes, or a general-purpose computer specially configured for the desired purpose by at least one computer program stored in a typically non-transitory computer-readable storage medium. The term “non-transitory” is used herein to exclude transitory, propagating signals or waves, but to otherwise include any volatile or non-volatile computer memory technology suitable to the application.

Any suitable processor/s, display and input means may be used to process, display, e.g., on a computer screen or other computer output device, store, and accept information such as information used by or generated by any of the methods and apparatus shown and described herein; the above processor/s, display and input means including computer programs, in accordance with all or any subset of the embodiments of the present invention. Any or all functionalities of the invention shown and described herein, such as but not limited to operations within flowcharts, may be performed by any one or more of: at least one conventional personal computer processor, workstation or other programmable device or computer or electronic computing device or processor, either general-purpose or specifically constructed, used for processing; a computer display screen and/or printer and/or speaker for displaying; machine-readable memory such as flash drives, optical disks, CDROMs, DVDs, BluRays, magnetic-optical discs or other discs; RAMs, ROMs, EPROMS, EEPROMs, magnetic or optical or other cards, for storing, and keyboard or mouse for accepting. Modules illustrated and described herein may include any one or combination or plurality of: a server, a data processor, a memory/computer storage, a communication interface (wireless (e.g., BLE) or wired (e.g., USB)), a computer program stored in memory/computer storage.

The term “process” as used above is intended to include any type of computation or manipulation or transformation of data represented as physical, e.g. electronic, phenomena which may occur or reside e.g. within registers and/or memories of at least one computer or processor. Use of nouns in singular form is not intended to be limiting; thus the term processor is intended to include a plurality of processing units which may be distributed or remote, the term server is intended to include plural typically interconnected modules running on plural respective servers, and so forth.

The above devices may communicate via any conventional wired or wireless digital communication means, e.g., via a wired or cellular telephone network, or a computer network such as the Internet.

The apparatus of the present invention may include, according to certain embodiments of the invention, machine readable memory containing or otherwise storing, a program of instructions, which, when executed by the machine, implements all or any subset of the apparatus, methods, features, and functionalities of the invention shown and described herein. Alternatively, or in addition, the apparatus of the present invention may include, according to certain embodiments of the invention, a program as above which may be written in any conventional programming language, and optionally a machine for executing the program, such as but not limited to a general-purpose computer which may optionally be configured or activated in accordance with the teachings of the present invention. Any of the teachings incorporated herein may, wherever suitable, operate on signals representative of physical objects or substances.

The embodiments referred to above, and other embodiments, are described in detail in the next section.

Any trademark occurring in the text or drawings is the property of its owner and occurs herein merely to explain or illustrate one example of how an embodiment of the invention may be implemented.

Unless stated otherwise, terms such as, “processing”, “computing”, “estimating”, “selecting”, “ranking”, “grading”, “calculating”, “determining”, “generating”, “reassessing”, “classifying”, “generating”, “producing”, “stereo-matching”, “registering”, “detecting”, “associating”, “superimposing”, “obtaining”, “providing”, “accessing”, “setting” or the like, refer to the action and/or processes of at least one computer/s or computing system/s, or processor/s or similar electronic computing device/s or circuitry, that manipulate and/or transform data which may be represented as physical, such as electronic, quantities, e.g., within the computing system's registers and/or memories, and/or may be provided on-the-fly, into other data which may be similarly represented as physical quantities within the computing system's memories, registers or other such information storage, transmission or display devices or may be provided to external factors e.g. via a suitable data network. The term “computer” may be broadly construed to cover any kind of electronic device with data processing capabilities, including, by way of non-limiting example, personal computers, servers, embedded cores, computing systems, communication devices, processors (e.g., digital signal processors (DSPs), microcontrollers, field programmable gate arrays (FPGAs), application specific integrated circuits (ASICs), etc.) and other electronic computing devices. Any reference to a computer, controller, or processor, is intended to include one or more hardware devices, e.g., chips, which may be co-located or remote from one another. Any controller or processor may, for example, comprise at least one CPU, DSP, FPGA or ASIC, suitably configured in accordance with the logic and functionalities described herein.

Any feature or logic or functionality described herein may be implemented by processor/s or controller/s configured as per the described feature or logic or functionality, even if the processor/s or controller/s are not specifically illustrated for simplicity. The controller or processor may be implemented in hardware, e.g., using one or more Application-Specific Integrated Circuits (ASICs) or Field-Programmable Gate Arrays (FPGAs) or may comprise a microprocessor that runs suitable software, or a combination of hardware and software elements.

The present invention may be described, merely for clarity, in terms of terminology specific to, or references to, particular programming languages, operating systems, browsers, system versions, individual products, protocols and the like. It will be appreciated that this terminology or such reference/s is intended to convey general principles of operation clearly and briefly, by way of example, and is not intended to limit the scope of the invention solely to a particular programming language, operating system, browser, system version, or individual product or protocol. Nonetheless, the disclosure of the standard or other professional literature defining the programming language, operating system, browser, system version, or individual product or protocol in question, is incorporated by reference herein in its entirety.

Elements separately listed herein need not be distinct components, and alternatively may be the same structure. A statement that an element or feature may exist is intended to include (a) embodiments in which the element or feature exists; (b) embodiments in which the element or feature does not exist; and (c) embodiments in which the element or feature exist selectably, e.g., a user may configure or select whether the element or feature does or does not exist.

Any suitable input device, such as but not limited to a sensor, may be used to generate or otherwise provide information received by the apparatus and methods shown and described herein. Any suitable output device or display may be used to display or output information generated by the apparatus and methods shown and described herein. Any suitable processor/s may be employed to compute or generate or route, or otherwise manipulate or process information as described herein and/or to perform functionalities described herein and/or to implement any engine, interface, or other system illustrated or described herein. Any suitable computerized data storage, e.g., computer memory, may be used to store information received by or generated by the systems shown and described herein. Functionalities shown and described herein may be divided between a server computer and a plurality of client computers. These or any other computerized components shown and described herein may communicate between themselves via a suitable computer network.

The system shown and described herein may include user interface/s e.g. as described herein, which may, for example, include all or any subset of: an interactive voice response interface, automated response tool, speech-to-text transcription system, automated digital or electronic interface having interactive visual components, web portal, visual interface loaded as web page/s or screen/s from server/s via communication network/s to a web browser or other application downloaded onto a user's device, automated speech-to-text conversion tool, including a front-end interface portion thereof and back-end logic interacting therewith. Thus, the term user interface or “UI” as used herein includes also the underlying logic which controls the data presented to the user, e.g., by the system display, and receives and processes and/or provides to other modules herein, data entered by a user, e.g., using her or his workstation/device.

### BRIEF DESCRIPTION OF THE DRAWINGS

Certain embodiments of the present invention are illustrated in the following drawings; in the block diagrams, arrows between modules may be implemented as APIs, and any suitable technology may be used for interconnecting functional components or modules illustrated herein in a suitable sequence or order, e.g., via a suitable API/Interface. For example, state of the art tools may be employed, such as but not limited to Apache Thrift and Avro which provide remote call support. Or, a standard communication protocol may be employed, such as but not limited to HTTP or MQTT, and may be combined with a standard data format, such as but not limited to JSON or XML. According to one embodiment, one of the modules may share a secure API with another module. Communication between modules may comply with any customized protocol or customized query language, or may comply with any conventional query language or protocol.

Specifically:

FIG. 1 is a simplified block diagram illustration of a system for remote passive monitoring of visual health.

FIGS. 2 and 3 are simplified flowchart illustration of methods (e.g. for operating the system of FIG. 1) in accordance with certain embodiments herein which may be used separately or together. For example, FIG. 2 may be used to perform screening/detection in accordance with operation/s 340 and/or 350 of FIG. 3.

Methods and systems included in the scope of the present invention may include any subset or all of the functional blocks shown in the specifically illustrated implementations by way of example, in any suitable order, e.g., as shown. Flows may include all or any subset of the illustrated operations, suitably ordered, e.g., as shown. Tables herein may include all or any subset of the fields and/or records and/or cells and/or rows and/or columns described.

Computational, functional or logical components described and illustrated herein may be implemented in various forms, for example as hardware circuits, such as but not limited to custom VLSI circuits or gate arrays or programmable hardware devices such as but not limited to FPGAS, or as software program code stored on at least one tangible or intangible computer-readable medium and executable by at least one processor, or any suitable combination thereof. A specific functional component may be formed by one particular sequence of software code, or by a plurality of such, which collectively act or behave or act as described herein with reference to the functional component in question. For example, the component may be distributed over several code sequences, such as but not limited to objects, procedures, functions, routines, and programs, and may originate from several computer files which typically operate synergistically.

Each functionality or method herein may be implemented in software (e.g. for execution on suitable processing hardware such as a microprocessor or digital signal processor), firmware, hardware (using any conventional hardware technology such as Integrated Circuit technology) or any combination thereof.

Functionality or operations stipulated as being software-implemented may alternatively be wholly or fully implemented by an equivalent hardware or firmware module, and vice-versa. Firmware implementing functionality described herein, if provided, may be held in any suitable memory device, and a suitable processing unit (aka processor) may be configured for executing firmware code. Alternatively, certain embodiments described herein may be implemented partly or exclusively in hardware, in which case all or any subset of the variables, parameters, and computations described herein may be in hardware.

Any module or functionality described herein may comprise a suitably configured hardware component or circuitry. Alternatively or in addition, modules or functionality described herein may be performed by a general purpose computer, or more generally by a suitable microprocessor, configured in accordance with methods shown and described herein, or any suitable subset, in any suitable order, of the operations included in such methods, or in accordance with methods known in the art.

Any logical functionality described herein may be implemented as a real time application, if, and as appropriate, and which may employ any suitable architectural option, such as but not limited to FPGA, ASIC, or DSP, or any suitable combination thereof.

Any hardware component mentioned herein may in fact include either one or more hardware devices, e.g., chips, which may be co-located or remote from one another.

Any method described herein is intended to include, within the scope of the embodiments of the present invention, also any software or computer program performing all or any subset of the method's operations, including a mobile application, platform or operating system, e.g., as stored in a medium, as well as combining the computer program with a hardware device to perform all or any subset of the operations of the method.

Data may be stored on one or more tangible or intangible computer readable media stored at one or more different locations, different network nodes or different storage devices at a single node or location.

It is appreciated that any computer data storage technology, including any type of storage or memory and any type of computer components and recording media that retain digital data used for computing for an interval of time, and any type of information retention technology, may be used to store the various data provided and employed herein. Suitable computer data storage or information retention apparatus may include apparatus which is primary, secondary, tertiary, or off-line; which is of any type or level or amount or category of volatility, differentiation, mutability, accessibility, addressability, capacity, performance and energy use; and which is based on any suitable technologies such as semiconductor, magnetic, optical, paper, and others.

Methods and systems included in the scope of the present invention may include any subset or all of the functional blocks shown in the specifically illustrated implementations by way of example, in any suitable order, e.g., as shown. Flows may include all or any subset of the illustrated operations, suitably ordered, e.g., as shown. Tables herein may include all or any subset of the fields and/or records and/or cells and/or rows and/or columns described.

Computational, functional or logical components described and illustrated herein may be implemented in various forms, for example as hardware circuits, such as but not limited to custom VLSI circuits or gate arrays or programmable hardware devices such as but not limited to FPGAS, or as software program code stored on at least one tangible or intangible computer-readable medium and executable by at least one processor, or any suitable combination thereof. A specific functional component may be formed by one particular sequence of software code, or by a plurality of such, which collectively act or behave or act as described herein with reference to the functional component in question. For example, the component may be distributed over several code sequences, such as but not limited to objects, procedures, functions, routines, and programs, and may originate from several computer files which typically operate synergistically.

Each functionality or method herein may be implemented in software (e.g. for execution on suitable processing hardware such as a microprocessor or digital signal processor), firmware, hardware (using any conventional hardware technology such as Integrated Circuit technology) or any combination thereof.

Functionality or operations stipulated as being software-implemented may alternatively be wholly or fully implemented by an equivalent hardware or firmware module, and vice-versa. Firmware implementing functionality described herein, if provided, may be held in any suitable memory device, and a suitable processing unit (aka processor) may be configured for executing firmware code. Alternatively, certain embodiments described herein may be implemented partly or exclusively in hardware, in which case all or any subset of the variables, parameters, and computations described herein may be in hardware.

Any module or functionality described herein may comprise a suitably configured hardware component or circuitry. Alternatively or in addition, modules or functionality described herein may be performed by a general purpose computer, or more generally by a suitable microprocessor, configured in accordance with methods shown and described herein, or any suitable subset, in any suitable order, of the operations included in such methods, or in accordance with methods known in the art.

Any logical functionality described herein may be implemented as a real time application, if, and as appropriate, and which may employ any suitable architectural option, such as but not limited to FPGA, ASIC, or DSP, or any suitable combination thereof.

Any hardware component mentioned herein may in fact include either one or more hardware devices, e.g., chips, which may be co-located or remote from one another.

Any method described herein is intended to include, within the scope of the embodiments of the present invention, also any software or computer program performing all or any subset of the method's operations, including a mobile application, platform or operating system, e.g., as stored in a medium, as well as combining the computer program with a hardware device to perform all or any subset of the operations of the method.

Data may be stored on one or more tangible or intangible computer readable media stored at one or more different locations, different network nodes or different storage devices at a single node or location.

It is appreciated that any computer data storage technology, including any type of storage or memory and any type of computer components and recording media that retain digital data used for computing for an interval of time, and any type of information retention technology, may be used to store the various data provided and employed herein. Suitable computer data storage or information retention apparatus may include apparatus which is primary, secondary, tertiary, or off-line; which is of any type or level or amount or category of volatility, differentiation, mutability, accessibility, addressability, capacity, performance and energy use; and which is based on any suitable technologies such as semiconductor, magnetic, optical, paper, and others.

### DETAILED DESCRIPTION OF CERTAIN EMBODIMENTS

It is advantageous to monitor the progression of a person's visual deterioration, at least for all or any subset of the following reasons:
1. Early detection of potential eye conditions such as but not limited to glaucoma, macular degeneration, diabetic retinopathy, or cataracts. It is appreciated that early diagnosis of some conditions may be crucial, e.g. if the eye condition is more treatable when caught in the early stages, helping to slow progression or even prevent complete vision loss.
2. Tracking disease progression, e.g. if a condition is deteriorating rapidly, more aggressive treatments may be recommended or scheduled interventions, e.g. surgery, which were defined as non-urgent and scheduled accordingly, may be automatically re-defined as urgent, and, accordingly, re-scheduled earlier.
3. Supplying supportive hardware or technology: monitoring deterioration may help time adaptations for a user's environment, e.g., if a person's vision is found by gait analysis to have worsened to below a given threshold stored in the system, the user may be supplied with, or prompted to equip herself or himself with, suitable hardware e.g. increased lighting, assistive technologies, or accident prevention solutions.
4. Monitoring driving ability: users may be prompted to cease driving if their gait analysis shows that their vision has worsened to below a given threshold stored in the system.
5. Recommending lifestyle changes (such as better nutrition or at-home exercises), or low-vision rehabilitation, or fall prevention.

Thus, monitoring for visual impairment, as conventionally practiced, is well-established as a critical aspect of healthcare, yet, to date, monitoring is envisaged in terms of periodical in-clinic eye examinations, and no more. Conventional visual ability monitoring typically takes place only in the clinic. Eye doctor appointments occur, at best, annually or (for persons with a diagnosed eye condition) twice-yearly.

Unfortunately, the inherent disadvantages in conventional monitoring for visual impairment, e.g. cost, ability to cooperate as a requirement, and less than 100% compliance, have to date been accepted as a fact, and have not been targeted as a goal for research and development.

Certain embodiments herein seek to provide improved methods for monitoring visual ability.

It is appreciated that some eye conditions, e.g. glaucoma, diabetic retinopathy, age-related macular degeneration (AMD), and cataracts, are more easily and/or effectively treatable when caught early, thus speed of detection may be advantageous for preserving vision and preventing permanent damage. Even early detection of Retinitis Pigmentosa (RP) is considered important. Typically, the visual deterioration caused by any damaging eye condition, including, but not limited to the above, is detected e.g. as described herein, and a human expert then determines which of the above (or other) is the correct diagnosis, typically in an in-clinic visit.

Optionally, the system may get outcomes, e.g. any diagnosis of the above five conditions by a human doctor) and use this as tagged data to learn enough to suggest a diagnosis for consideration by a human expert, even based on IMU data alone, e.g. one of the above five conditions. For example, certain conditions may affect the patient in daytime or morning and night differently, which may enable distinguishing between these conditions when comparing gait mobility measures at different times during the day. There may turn out to be other distinguishing features in the visual deterioration caused by these conditions, which are detectable through gait analysis, and thus may be machine-learned as tagged data accumulates.

For example, when detected early, glaucoma may be managed with medication (eye drops), laser therapy, or surgery, all of which may slow or halt progression of the disease and prevent further vision loss. Optimal detection time is before significant nerve damage occurs, which is why regular eye exams are crucial, given early-stage glaucoma may be asymptomatic, and detection involves in-clinic detection of elevated intraocular pressure or early signs of optic nerve damage, despite no noticeable vision loss. According to embodiments herein, the system machine-learns how to estimate intraocular pressure based on gait analysis, thus without, or before, an end-user suffering from elevated IOP reaches a clinic for routine evaluation.

According to an embodiment of the invention, the system receives, from an organization e.g. HMO aka Health Maintenance Organization served by the system which provides health services to end-users bearing cellphones, indications that certain individuals have been diagnosed with eye conditions causing visual deterioration, such as but not limited to glaucoma, diabetic retinopathy, age-related macular degeneration (AMD), cataracts, and Retinitis Pigmentosa (RP). The purpose of these indications may be to use a gait analysis-based system, e.g. as described herein, to passively and repeatedly analyze the IMU data generated by each end-user's cellphone, and, using this, to help with disease management, for any disease which affects mobility, and slow its progression by providing more proactive care by responding to indications of gait deterioration identified by gait analysis of passively measured IMU data, where “passive” refers to IMU data collection which does not require user cooperation each time the IMU data is collected.

The system, responsive to these indications, typically monitors disease progression in the diagnosed individuals, based on gait analysis, e.g. as described herein, and, using any gait parameter or combination thereof, e.g. as described herein. The system may, for example, learn to estimate stages in the above conditions, if human expert-generated staging data is provided regarding some end-users.

The system may also learn to provide early diagnosis of the above conditions by machine-learning IMU data generated by the diagnosed individuals' phones at a point in time prior to their diagnosis date, e.g. 6 months or 12 months before each end-user's diagnosis date. The system may alternatively, or in addition, learn gait parameters e.g. any of those described herein generated from the IMU data generated by the diagnosed individuals' phones at a point in time, prior to their diagnosis date. For example, IMU data may be collected from a healthy population and also from a diagnosed population, at a time point which precedes by six months this population's date of diagnosis, typically say 50 people in each of the two groups, and, using this as training data, a classifier may be trained to predict to which group a given end-user belongs: the healthy group, or the group which may likely be diagnosed with one of the above conditions, within (say) six months.

It is appreciated that in-clinic, optic nerve damage, or increased eye pressure, may be identified with a tonometry test to measure eye pressure, along with a visual field test to assess peripheral vision, although these tests require varying degrees of cooperation. If untreated, glaucoma may lead to progressive and irreversible loss of peripheral vision, and even complete blindness, if complete optic nerve damage has been allowed to occur.

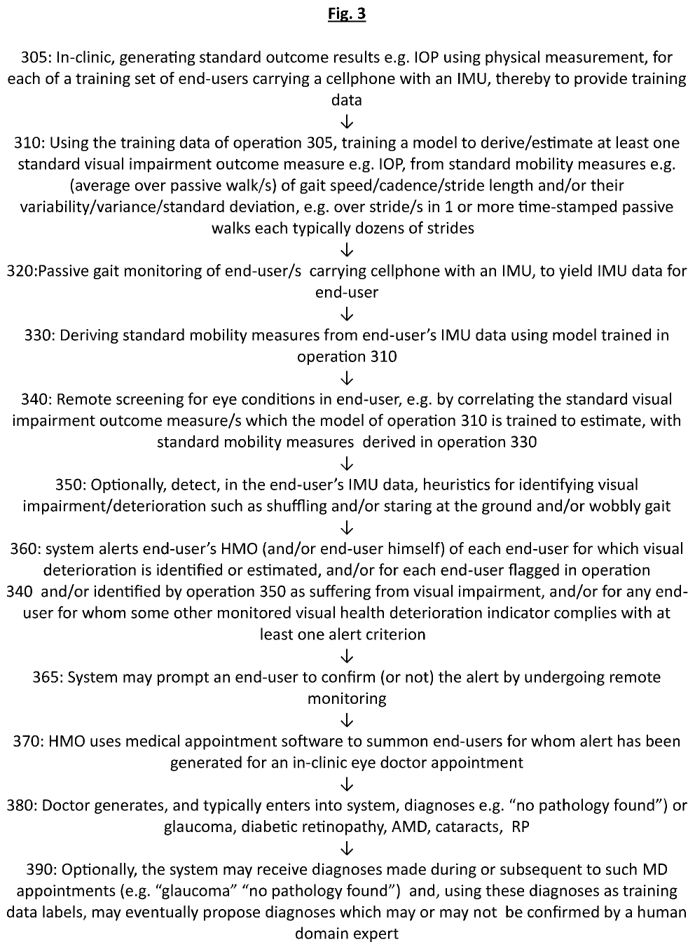

It is appreciated that gait analysis may be fine-tuned to predict the need to carry out urgent tonometry e.g. by using balance and variability decline. Any suitable gait parameters may be used to measure this decline e.g. (in static balance measurement) duration of balance and magnitude of movement and/or during walking which may be passively measured, all or any subset of gait speed, stride length, variability, stride width, under different light conditions, to detect visual deterioration. A model may be trained, e.g. as described in FIG. 2 herein, that predicts tonometry levels (IOP-intraocular pressure), using mobility measures, to detect IOP changes through changes in mobility measures. Typically, each time the system detects a risk of IOP increase for a given end-user, e.g. each time the system identifies an end-user whose estimated IOP has risen, the end-user and/or at least one entity involved in the end-user's care, e.g. her or his health organization, receives an alert.

The system of the present invention may include all or any subset of the following three components:
1. A patient-facing mobile app aka patient app—this component enables the system to collect data from the patient, as well as communicate back to the patient to provide clinical value and encourage the patient to be engaged and adhere to their care program. This component is described in detail in the section on the patient's app.
2. A caregiver dashboard—this component allows the clinician to take different actions of care based on the patient's data for the individual patient. Moreover, it enables the management of clinical resources such as clinician attention and triage of patients in the scope of a clinic having a group of patients. Furthermore, a clinic manager or an organizational manager may compare different clinics in terms of mobility assessment of their patients to make organizational decisions. These three levels of data-driven decisions are elaborated on in the section on the caregiver's dashboard.
3. A digital care web service—this component is responsible for communication with the two other components, and enables their functionalities. In addition, it is responsible for analysis of the data collected from the phone's imu.

The patient app serves as an endpoint of the system on the patient's side, and may be configured to enable all or any subset of: collection of patient data, reflection of the patient's status, and communication with the caregiver and home program. Reflection of the patient's status increases their engagement with the app, which, in turn, increases the data available for the system; a patient engaged with the app e.g. because s/he sees her/his walk score which may include a composite measure reflecting how their visual condition impacts mobility gains value, becomes more engaged and is likely to be willing to take measurements and/or complete questionnaires, e.g. to take another walk measurement to verify the test, or to undergo a visual test, via the app.

The patient app collects objective gait analysis which may include all or any subset of the following measures: a. Spatiotemporal analysis: evaluation of gait properties such as gait cycle time, cadence, gait speed, stride length, right and left step length, base width, right and left single support and stance time and percentage, double support time and percentage.
b. Kinematics: including any of the lower body joint angles over the gait cycle.
c. Kinetic: evaluation of gait properties related to torques and forces such as ground forces and center of feet pressure over the gait cycle, and the analysis of its sway over the gait cycle.
d. Variability: deviation of any of the gait properties above between different strides over the same walk. May be processed in two forms, first aggregated as the standard deviation of each value, and second as a sequence of the differences in values over time.
   Typically, periodically (e.g. every day at 12 noon system time) or triggered by a patient's activity (e.g. any communication with the service such as a mobility measurement) or by a caregiver's activity (e.g. any communication with the service) the system starts the monitoring flow for the corresponding patient or patients (when it is periodical or initiated by a caregiver treating a group of patients). Once the monitoring flow starts for a certain patient, the service first retrieves and gathers all of that patient's data, then structures the patient's data, e.g. including filtering of noise and/or outliers, and eventually assesses the patient's current status expressed as a set of indications and their different types that comprise the patient's status.
   Patient data over time may include all or any subset of:
   Patient's demographic and clinical condition
   Objective gait data e.g. via gait analysis
   Standard test outcome measures e.g. as estimated from gait analysis
   Subjective data
   Clinical events.

The structure of a patient's longitudinal representation aka structuring process may include outlier filtering and/or interpolation of trends.
Typically, the system generates indication alerts that contain information on the patient's status and progress in relation to visually impairment on any given date. As input, the system typically takes into account the structured longitudinal patient data. This data typically includes spatiotemporal gait parameters which may be measured with OneStep's mobile gait assessment technology (including but not limited to all or any subset of the following gait parameters: velocity; step and stride length; double support, single support, stance; measures of asymmetry such as stance asymmetry and/or step length asymmetry; and measures of variability such as cadence variability and/or velocity variability). This data may also include results of standard tests (such as timed up-and-go and sit-to-stand tests, and/or patient reported outcomes, and/or kinetic and/or kinematic measures of gait e.g. as measured with OneStep's smartphone-based technology. From this data, the system typically assesses the patient's status based on a comparison to established norms and/or values found within the literature of research on visual impairment, and/or on data collected (e.g. by OneStep) regarding patients with visual impairment. This status may contain information on the level of improvement and/or decline compared to a baseline point in time, and may explain or document the information that went into the decision-making process for generating the status indication. Such indications may also contain suggested therapeutic interventions tailored to the patient e.g. based on the longitudinal data of that patient.

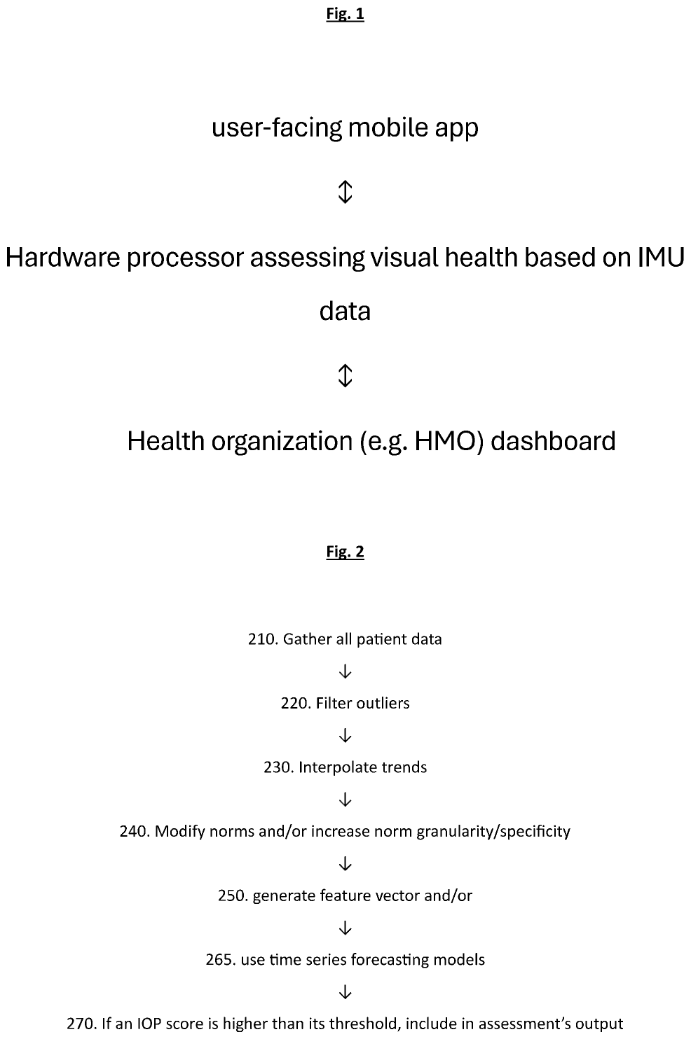

FIG. 1 illustrates a system for remote passive monitoring of visual health which includes a mobile app and/or a health organization dashboard, and/or a hardware processor configured to receive IMU data from registered users' cellphones and/or gait analysis derived therefrom, and to communicate with the user-facing mobile app and/or the health organization dashboard. Typically, the processor continuously (e.g. more frequently than traditional once-or-twice-yearly in-clinic visits) assesses the mobile app user's visual health status and interacts according with the user and/or health organization, via the app/dashboard respectively.

The system of FIG. 1 may for example be used to, repeatedly:
1. Start assessment of a given end-user bearing a cellphone with built in IMU
2. Gather all patient information e.g. all or any subset of: Mobility data, Norms, Subjective data, Clinical events
And 3. Assess patient's current visual health status e.g. to detect Visual deterioration

Reference is now made to FIG. 3 which illustrates an example method of operation e.g. for the system of FIG. 1, all or any subset of whose operations may be performed in practice, in any suitable order e.g. as shown. The method of FIG. 3 may be used for data collection and/or for screening and/or for reporting.

Typically, operations 305, 310 are set-up operations performed offline in advance; operations 320 onward may be performed in run-time. The set-up operations are thus typically all or any subset of these:
Operation 305: In-clinic, generating standard outcome results e.g. IOP using physical measurement, for each of a training set of end-users carrying a cellphone with an IMU, thereby to provide training data
Operation 310: Using the training data of operation 305, training a model to derive, from at least one standard mobility measure e.g. gait speed and its variability/variance/standard deviation, and estimate at least one standard visual impairment outcome measure such as IOP

The run-time operations are thus typically all or any subset of these:
Operation 320: Passive gait monitoring of at least one end-user carrying a cellphone with an IMU, to yield IMU data for the end-user.
Operation 330: Deriving standard mobility measures from the end-user's IMU data.
Operation 340: Remote screening for new (or worsening) and possibly urgent eye conditions in the end-user, e.g. by correlating the standard visual impairment outcome measure/s which the model of operation 310 is trained to estimate, with the standard mobility measures derived in operation 330.
Operation 350: Optionally, detect, in the end-user's IMU data, heuristics for identifying visual impairment/deterioration such as shuffling and/or staring at the ground and/or wobbly gait.
Operation 360: For each end-user for which visual deterioration is identified or estimated, and/or for each end-user for whom operation 340 flags as having a new or worsening eye condition and/or for whom operation 350 identifies as suffering from visual impairment, and/or for any end-user for whom some other monitored visual health deterioration indicator complies with at least one alert criterion, the system alerts the end-user's HMO (and/or end-user himself).
Operation 365: System may prompt an end-user to confirm (or not) the alert by undergoing remote monitoring.
Operation 370: The HMO may, e.g. using medical appointment software, summon end-users for whom an alert has been generated for an in-clinic eye doctor appointment which may include an in-clinic session with a digital retinal camera and/or an in-clinic optical coherence tomography (OCT) session and/or visual acuity tests and/or tonometry and/or dilated eye exam.
Operation 380: Doctor generates, and typically enters into system, diagnoses e.g. “no pathology found”) or glaucoma, diabetic retinopathy, age-related macular degeneration (AMD), cataracts, or Retinitis Pigmentosa (RP).
Operation 390: Optionally, the system may receive diagnoses made during or subsequent to such MD appointments (e.g. “glaucoma” “no pathology found”) and, using these diagnoses as training data labels, may eventually propose diagnoses which may or may not be confirmed by a human domain expert, e.g. system may learn to provide early diagnosis of the above conditions by machine-learning IMU data generated by the diagnosed individuals' phones at a point in time prior to their diagnosis date.

The method of FIG. 2, via which a model may be trained that predicts tonometry levels (IOP-intraocular pressure), using mobility measures, to detect IOP changes through changes in mobility measures, is now described; this method may include all or any subset of the operations 210, 220, . . . described below, in any suitable order e.g. as illustrated in FIG. 2. It is appreciated that any of the teachings e.g. regarding standard outcome prediction described or illustrated in co-owned U.S. Ser. No. 19/049,400, filed Feb. 10, 2025, of which the current application is a continuation-in-part, the entire contents of which being hereby fully incorporated herein by reference, may be used to augment or replace all or any operations of the method of FIG. 2.

210. Gather all patient data-all information provided on the dashboard or using the apparatus e.g. all or any subset of the following: demographic information, clinical conditions, treatment log, active and passive gait analysis, standard mobility test analysis, patient-reported outcomes and questionnaires, clinical events, injuries, surgeries, and medication. This is typically performed on the cloud, whereas all or any subset of operations 220-270 may be performed locally.

220. Filter outliers which may be disrupted or noisy data; there may be some discrepancies in the estimated mobility measures, e.g. gait analysis measures and mobility test measures. This is even more likely in passive gait measurements, since environmental effects are unknown and may be challenging. To reduce such disrupted or noisy data, the system may filter out outliers (or at least indicate outliers with lower certainty or lower level of confidence). This task is possible e.g. when the patient's timeline is available, which enables comparison of data points and the patient's reference. For example, a gait speed 3 (say) standard deviations higher or lower than the patient's weekly average gait speed may be considered an outlier. When many data points are available, as in passive gait monitoring, a daily or weekly (or any other timespan) aggregation, taking the median or the average, may be used as a basis of comparison, to reduce outliers' noise.

230. Interpolate trends-Mobility measures (such as gait speed, stance asymmetry, ranges of motion, and PROs (patient-reported outcomes) typically have a characteristic behavior which is continuous over time, e.g. changes in these measures are not usually dramatic, and thus the system may interpolate between data points. For example, if an end-user has recovered from a condition, and his/her gait speed the previous week was 0.7, and this week is 0.9, then somewhere in between the gait speed may be assumed to have been 0.8, unless a dramatic event occurs (such as an injury). It is thus reasonable to interpolate some mobility measures between data points, as long as no medical incident has occurred, and produce trends for those measures. Linear interpolation between data points, e.g. of the data points collected in operation 210 (for each measure), and then filtered in operation 220, may be carried out, and then averaging the resulting interpolated data over a sliding window, to reduce measurement fluctuations afterward. The more frequent the measurements are, the more representative is the trend line. Gait active measurements may be taken between a few times a week and once every two weeks, depending on the natural progression of the patient's condition. The sliding window length may be determined accordingly to match the measurement frequency the patient is required in the program. For example, if a patient walks a few times a day, the sliding window may be a day or two-day window, so that it covers several measurements. If a patient fills out one questionnaire each week, the sliding window's length may be 2 to 4 weeks.

240. Modify norms and/or increase norm granularity/specificity: norms are aggregations of patients' data and timelines over a specific population to compare to the patient's measures, indicating whether the patient is above or below the norms, for example gait parameter norms by demographic data, such as normative values of gait speed by age and gender. Another example is the recovery pace in different parameters or outcome measures post-surgery, for instance benchmarks of 6-minute walk test distance by week for different surgeries and populations. Norms and benchmarks may be hardcoded, supported by literature, or computed by observations. Hence, when new data is collected and the method of FIG. 2 is executed, there is an opportunity to regenerate norms. This process may alternatively run after a large set of new timelines and patient data is collected. Norms may become as granular and specific as available, e.g., gait norms for Parkinson's patients by gender, age, height, and weight.

250. Generate Feature vector-a set of selected variables, e.g. the outputs of operations 210-240 composes a vector representing the patient's data at a specific timestamp (or date), in which every index allocates a specific variable. These variables are derived from any or all of the outcomes and measures of the gait analysis, questionnaires, standard mobility tests, demographics, and norms. One alternative is to define the variable value or to define a variable's value at a specific timestamp on which the variable was not measured, to assign the corresponding measure's/variable's interpolated value at the specified timestamp. Another alternative is to set the value to be the actual value as last measured; typically, the system may add a representation (e.g. number of days including fraction) of the time delta since this variable was last measured. The measurement's time may be embedded e.g. as described in arxiv.org/pdf/1907.05321 which describes providing a model-agnostic vector representation for time.

260. Estimate IOP aka pressure indication aka pressure score for the user e.g. by using pre-trained machine learning models. Any suitable method may be employed to train the models e.g. as described herein. The models appropriate for operation 260 may include regression models, such as but not limited to SVM, linear regression, neural network regression models, decision tree-based models, random forest, and XGBoost.

265. Alternative to operations 250 and 260, or in addition, and rather than describing the machine learning problem of generating a pressure score using a feature vector: vector→score as a regression task where operation 260 herein performs regression analysis predicting IOP from the feature vector of operation 250, and regarding the IOP prediction task as a time series forecasting that gets observations, each of which corresponds to a different timed measure or a static data point (gender, condition, etc.). In this case, every observation may include all or any subset of the time representation (0 when the variable has no timestamp, such as gender), the variable type representation, and the value representation, e.g. as described in arxiv.org/pdf/2109.12218. A model trained on this time series of observations is then applied to estimate the pressure score. The models appropriate for operation 865 include time series forecasting models, such as but not limited to RNN, LSTM, and transformer-based models.

270. If a pressure indication/score is higher than a system-stored threshold e.g. a normal threshold known in the medical arts, include the pressure indication/score in the assessment's output. Typically, if IOP is lower than threshold→no indication, e.g. as describedherein.

It is appreciated that automated prediction of standard outcomes may include generating time-stamped feature vectors for end-users in the system, each time-stamped feature vector for a given end-user Joe on a given date/time such as 3 Oct. 2024 including data points in the system for Joe (e.g. gait parameters or mobility measures of Joe's which were all measured on Mar. 10, 2024, or standard outcomes such as PROs actually generated by Joe on that date, or demographic variables or clinical events), and/or interpolated data points if no measured data point is available for that date e.g. an interpolated value of a given gait parameter which may be interpolated because this parameter was measured on 3 different dates previous to Mar. 10, 2024 and perhaps on date/s subsequent to Mar. 10, 2024 as well, albeit not on Mar. 10, 2024 itself.

Such feature vectors may be generated for plural end-users, and including for end-user David, for whom it is sought to generate an estimate of IOC or any other standard outcome, time-stamped for, say, Apr. 10, 2024. Typically the plural end-users also have IOC measurements in the system, which typically are not included in feature vectors generated for prediction of IOC. For example, user A may have an IOC measurement dated 1 July and user B may have an IOC measurement in the system dated 1 August. Then, a model (e.g. Random Forests or Gradient Boosting Machine or any suitable decision tree) may be trained to predict IOC (dependent variable), from feature vector/s (independent variable/s) whose time-stamps are, say, one day before that user's stored IOC value. Thus the feature vector generated for user A (to be paired with user A's 1 July's IOC measurement in the model's training data) may be from 30 June and the feature vector generated for user B (to be paired with user A's 1 August IOC measurement in the model's training data) may be from 31 July.

Alternatively, when seeking to estimate IOC or some other standard outcome, time-series models may be used directly on various data points (e.g. PRO's, gait parameters, clinic tests, demographic variables etc.) which are variously time-stamped, all of which typically belong to end-users in the system for whom measured IC results, variously time-stamped, are available. In this embodiment, no interpolation between existing dates in the system to yield a feature vector of identically-time-stamped data points is typically performed and instead, forecasting occurs directly from the variously time-stamped measured data available in the system (typically for end-users for whom measured IOC results, variously time-stamped, are available in the system).

According to certain embodiments, IMU data during a passive walk, or gait parameters derived therefrom, may be collected from a healthy population with normal IOP and also from a population of end-users known to have elevated IOP, typically say 50 people in each of the two groups, and, using this as training data, a classifier may be trained to predict to which group a given user U, for whom a given passive walk has been measured and analyzed, belongs, e.g. is the end-user U who performed the passive walk is healthy, i.e. with normal IOP, or is he predicted to have elevated IOP.

More generally, classifiers may be trained to predict or estimate any standard assessment of clinical outcome about which data is available to the system, such as but not limited to self-report patient questionnaires or PROs, e.g. questions relevant to visual health, clinician assessments, lab results, in-clinic measurements, disease-specific indexes, reported falls, etc.

A classifier may be trained to differentiate between cellphone accelerometer data which preceded in-clinic detection of a need to carry out urgent tonometry, from cellphone accelerometer data which preceded a clinic session which ruled out any need to carry out urgent tonometry. For example, data is collected from the population in which a need for testing was identified in-clinic, and also from a population of end-users for which no such need was identified in-clinic, typically say 50 people in each of the two groups, and, using this as training data, a classifier may be trained to predict to which group a given end-user belongs.

It is appreciated that by machine-learning accelerometer (IMU) data from phones of persons for whom tonometry or field of vision tests in-clinic successfully detected glaucoma at a very early stage, the system may identify combinations of gait parameter deterioration which predict this early detection, e.g. by training a classifier to distinguish between these cases, and between cases in which tonometry or field of vision tests detected no pathology.

It is appreciated that this embodiment is greatly advantageous, because once significant damage occurs, treatment may no longer be able to prevent further vision loss.

Similarly, diabetic retinopathy in its early or non-proliferative stages may be asymptomatic, but may alternatively show mild symptoms which may be detectable by gait analysis, as described herein. Strictly controlled blood sugar levels and/or laser therapy or injections of anti-VEGF (vascular endothelial growth factor) medications may then prevent or delay the progression of the disease.

The gait of persons suffering from impaired night vision typically differs from the gait of persons suffering from impaired vision generally, e.g. because the latter group exhibits somewhat worse gait parameters at night (relative to daytime), whereas the former group exhibits much worse gait parameters at night (relative to daytime). Thus the difference between gait parameter impairment during the night, relative to daytime in the same person, is more significant in persons suffering from impaired night vision relative to persons suffering from impaired vision generally, for which the difference between gait parameter impairment during the night, relative to daytime in the same person, is less significant.

It is appreciated that if an individual is found to suffer from deterioration of visual health, the individual's performance may be evaluated separately in time-windows known by the system to be hours of light, and in time-windows known by the system to be hours of dark, e.g. using the ambient light sensor which conventional cellphones have, and use, e.g. to adjust, the phone's screen brightness automatically, responsive to varying levels of ambient light and/or using time-stamps, a user's location, and stored data regarding times of sunrise and sunset in the user's location.

It is appreciated that when the environment is more challenging, e.g. there is less ambient light, visual impairment may become more easily distinguishable e.g. the impaired person's incompetence to overcome a low level of ambient light may differ more from the level of competence compared to a person who is not visually impaired, relative to the difference in competence between persons who are and are not visually impaired, in higher levels of ambient light.

It is appreciated that in-clinic retinal examination is the state-of-the-art best method for identifying early retinopathy. However, gait analysis may detect gait deterioration e.g. if vision has become blurry because of fluid leakage from damaged blood vessels in the retina, causing macular edema. Another sign useful for screening, especially in known diabetics, may be deterioration in gait during night hours, e.g. due to reduced night vision due to early retinopathy.

If these or other gait analysis outcomes suggest a suspicion of retinopathy, especially in known diabetics, the system may prompt the user to, via her or his phone, test for blurry or distorted vision in the central field by checking whether the user's ability to perform tasks such as reading or recognizing faces, has deteriorated relative to previous norms of her or his own, which may have been pre-stored in the system, particularly for known diabetics, or even relative to population norms.

The state-of-the-art recommendation to detect retinopathy early is simply yearly in-clinic dilated fundus photography, however considerably less than all affected individuals comply with this sometimes onerous recommendation. The consequences of resulting late detection may then include vision loss and retinal detachment and/or the need for invasive treatments, such as vitrectomy (surgical removal of the vitreous gel) or laser surgery.

It is appreciated that any method herein for detecting retinopathy may be used by any of the systems for monitoring diabetics described in co-owned U.S. Ser. No. 19/049,400, filed Feb. 10, 2025 and entitled “System, Method And Computer Program Product For Monitoring Diabetics, from which the present application claims continuation-in-part status and whose disclosure is hereby incorporated by reference herein in its entirety. For example, known diabetics may be screened for new appearances of retinopathy by processing imu data to detect visual impairment according to any method herein.

Moreover, age-related macular degeneration (AMD) which is a leading cause of vision loss in older adults and affects the central vision, presents in its early stages, with drusen (yellow deposits) in the retina which may be identified only in-clinic. However, AMD may also cause blurred vision which typically is so mild that sufferers do not notice or report significant vision loss. Gait analysis can, however, detect early gait deterioration caused by this blurred vision, e.g. if a classifier is trained to differentiate between cellphone accelerometer data which preceded successful in-clinic early detection of AMD, from cellphone accelerometer data which preceded a clinic session, which, e.g. via OCT (optical coherence tomography) scans, ruled out any drusen or other changes in the macula.

It is appreciated that “accelerometer” and “IMU” may be interchanged in the present disclosure.

Once detected, the system may recommend treatment for this early AMD e.g. dietary changes, antioxidants, or injections of anti-VEGF medications to prevent further damage to the macula.

It is appreciated that Amsler grid tests may be used to detect distortions in vision, and may be administered via phone, thus a mobile phone user may be prompted to undergo Amsler testing remotely, without a clinic visit, to corroborate (or not) the concern that this end-user may have AMD. Thus, any end-user classified by the above classifier as being characteristic of end-users in which AMD was early-detected, may be prompted to undergo this, whereas any end-user who is not classified by the above classifier as being characteristic of end-users in which AMD was early-detected, may not be prompted to undergo this Amsler testing.

This embodiment is advantageous at least because late-detected AMD may progress to neovascular AMD, which, being more difficult to treat, may lead to severe central vision loss, which may rob a patient of his abilities in certain activities, such as reading and driving.

Moreover, cataracts can, if detected early, often be managed with glasses or stronger prescription lenses. However, if the cataract becomes more severe due to late detection, the only treatment is surgical removal of the clouded lens, which is invasive. The state-of-the-art method for early detection involves regular in-clinic eye exams, however compliance of patients in scheduling these is less than 100%, even for patients who are able to cooperate, and may be impossible for patients whose general state of health makes cooperation difficult or impossible. The longer cataracts remain undetected, hence are left untreated, the more complex the surgery may become, which may lead to permanent vision impairment.

Monitoring for visual impairment, as conventionally practiced, is well-established as a critical aspect of healthcare, yet, to date, monitoring is envisaged in terms of periodical in-clinic eye examinations, and no more. The system herein may prompt a user and/or his eye physician to schedule an appointment in-clinic, responsive to gait analysis as described herein, which then results in gait analysis generating indicators which may be used for screening. The recommended eye exams may include all or any subset of visual acuity tests: Tonometry and dilated eye exams to allow the doctor to see the retina, optic nerve, and blood vessels at the back of the eye, to identify macular degeneration, diabetic retinopathy, or retinal detachment, if present.

It is appreciated that at-home monitoring tools exist for Amsler grid and home tonometers, if available for certain end-users, such tools measuring intraocular pressure from home. The system herein may prompt a user to avail herself or himself of these, if so indicated by results indicative of reduced visual functioning obtained from gait analysis as described herein.

It is appreciated that when a user arrives at a clinic, health professionals may ask the user to report changes in activities such as reading, driving, or recognizing faces. This feedback may then be used in adjusting treatments or considering interventions such as low-vision aids or surgery. But, according to embodiments herein, the system may prompt the user to interact with her or his phone to directly test the user's proficiency in reading, driving, or recognizing faces, relative to system-stored population norms or relative to a pre-stored baseline stored from an individual by the system. This results in more accurate results, which may be obtained at home, rather than first being reported at the clinic, and which become available as soon as the system identifies a potentially worrying indication of deteriorating gait and the user complies with the system recommendation, rather than becoming available at best, only when the user happens to present herself or himself at a clinic, e.g. once per year.

The system is typically configured to extract standard, objective gait measures, typically including spatiotemporal parameters, such as but not limited to all or any subset of gait speed, cadence, stride length, temporal asymmetry, and their respective stride-to-stride variabilities, as well as kinetics and kinematics. This typically encompasses patterns such as high-knee walking, which may be quantified using spatiotemporal parameters such as foot clearance (the maximum height of the foot above the ground), and/or kinematics such as hip and/or knee flexion-extension. These or any other suitable mobility measures may then be correlated with, or used to predict, PROMs (or other standard outcome measures) which are known in the medical arts to be indicative of visual impairment. Such PROMs include, by way of non-limiting example, the National Eye Institute Visual Function Questionnaire (NEI-VFQ-25), Impact of Vision Impairment (IVI) Questionnaire, Low Vision Quality of Life (LVQOL) Questionnaire. Clinical objective measures aka tests known in the medical arts to be indicative of visual impairment include Intraocular Pressure (IOP), Reading Speed Tests, Orientation and Mobility (O&M) Tests.

It is appreciated that the method of FIG. 2 herein is, by way of example, directed to estimation of IOP. However, the method of FIG. 2 may more generally be used to predict other standard outcome measures) which are known in the medical arts to be indicative of visual impairment, such as but not limited to those specifically listed above.

Optionally, any suitable gait analysis may be employed herein, given that as a person becomes gradually more visually impaired, that is likely (e.g. because visual input is reduced, the brain must rely more on other sensory systems such as touch, proprioception, and hearing to control and adjust movement) to affect various aspects of her or his motor behavior, such as but not limited to all or any subset of the following:
a. Decreased coordination and balance, e.g. when walking on uneven surfaces. The system may collect data indicating which geographical locations have in the past elicited poor quality gait in previous users as evaluated when in these locations, may then assume these locations are uneven or otherwise challenging, and may then turn on gait evaluation automatically and passively, when users who need to be evaluated, arrive at such locations. Gait asymmetries and variability in stance durations and step length, compared in different environmental conditions, may also be used for this purpose.
b. Balance difficulties, e.g. when walking or even simply standing or walking. This may result in either or both of instability or a tendency to lean to one side.

According to certain embodiments, an end-user, e.g. one suspected of having balance difficulties and/or postural instability, may be prompted to take a static balance test, in a home setting, e.g. not requiring a clinical visit, and the system may then derive duration of balanced posture, and/or magnitude of movement.
c. Slower gait e.g. because reaction times may slow down (slower gait speed e.g.). The system may invite certain end-users to take a mobility reaction time test: the patient is instructed to start walking and requested to turn and go back in the other direction, e.g. by voice instructions from the app. The system may then measure the time from the instruction to the first step toward the new direction.
d. Shortened stride, e.g. the system may be configured to extract stride length using gait analysis, and to alert if an end-user's stride length shortens relative to his own stride length norm which may be stored in the system, or relative to population stride length norms.
e. Increased tripping over obstacles which may be picked up by gait analysis of the IMU data as a short interval of “shakes” (e.g. a few minutes duration, as opposed to a few hours).

It is appreciated that co-owned U.S. patent application Ser. No. 18/975,890 “Advanced Pedestrian Navigation Based on Inertial Gait Analysis and GPS Data”, incorporated by reference herein in its entirety, of which this application is a continuation-in-part, and/or U.S. patent application Ser. No. 17/500,744 published as US 2022/0111257, dated 13 Oct. 2021 and entitled “Efficient System Configured To Facilitate Physical Rehabilitation” also fully incorporated herein by reference, describe inter alia how to partition motion, measured using an IMU, into intervals characterized or classified using types such as but not limited to an interval of “shake/s”, and other types of intervals such as “walk intervals”, and “Device Transition” referring to a change of the bodily position of the measurement device, e.g. from the user's pocket to her or his hand.

The system may, for example, identify intervals of shakes, e.g. as described in co-owned U.S. Ser. No. 18/975,890 and/or 17/500,744, the entire contents of each of which are hereby fully incorporated herein by reference, and may then determine whether some or each of these are attempts of continued steps/strides, e.g. an interval of tripping and/or subsequent recovery from tripping, as opposed to other activities which may be categorized as shakes (e.g., bouncing up and down or dancing). To make this determination, the system may take all short (<say 3 seconds) shakes that occur when the phone is in the pocket which occur between sequences of strides e.g. walk intervals. The system typically knows the phone's bodily positions (thus knows when a user's phone is in her/his pocket) during/from the stride intervals (e.g. as described in co-owned U.S. Ser. No. 16/659,832 published as US 2020/0289027, entitled “Assessment Of A User's Gait” and dated 22 Oct. 2019, the entire contents of which being hereby fully incorporated herein by reference. The system also typically knows that no transition (no change of the phone's bodily position e.g. no transfer of phone from, say, left hip pocket to shirt pocket) happens before or after the shake e.g. because the shake interval occurred between two gait/stride sequences without any intervening interval of the “device transition” type. Such shake intervals may be collected from a healthy population and also from a population of end-users known to be unstable (e.g. at least 100 shakes for an individual), typically, say, 50 people in each of the two groups, and, using this as training data, a classifier may be trained to predict to which group a shake belongs, e.g. whether the end-user is healthy or unstable. It is appreciated that gait analysis of “shakes” may identify a pattern of variability in cadence and/or stride length which is not typically present in intervals of other types.

Thus, tripping may generate a different sequence of IMU data than an intentional change in gait of the same duration, e.g. because the tripping event may be followed by a time-period in which the end-user regains his stability, whereas the intentional change in gait would not be followed by such a time-period.

An example machine learning method may include all or any subset of the following operations, suitably ordered e.g. as follows:
1. Collect IMU data passively, from 100 healthy people walking in real-world settings and 100 people known (e.g. by their cohort, caregiver, or from experimental settings in which persons known to have various health conditions are studied) to be at risk of falls.
2. Identify 100 short (e.g. a few seconds, such as 2 or 3 or 5 seconds) intervals of shakes in the IMU data collected for each subject in both groups.
3. Train a classifier whose input is one of the identified shakes intervals, and which generates an output indicating whether the shakes interval belongs to the healthy group or the high-fall-risk group. Typically, “shakes” intervals are a result of an intentional activity such as dancing or jumping in healthy end-users, and are a result of tripping in high-fall-risk end-users.
4. Use classifier as trained to classify time-intervals of IMU data, online or in real-time, as tripping or not-tripping.
f. Gait slower than population norms in areas with traffic noise, e.g. busy streets known to the system, or areas of congestion (whose location may also be known to the system) in which typically many people are talking, since the person is more reliant than average on sound cues which are impeded by the noise or conversation in the area.
g. Postural changes to maintain stability, such as shifting body weight or rigid posture, minimizing spontaneous or subtle movements to reduce the risk of falling. It is appreciated that any known gait analysis may be employed to derive kinematics data for each user from her or his passive walks, repeatedly e.g. daily, thereby to provide a timeline of postural changes where kinematics which typically includes a formal definition of the user's joint angles e.g. in her or his lower body, is used as a representation of the user's posture. any suitable gait analysis may be employed for this purpose e.g. as described in U.S. Ser. No. 17/666,180 published as US 2023/0137198, dated Apr. 5, 2023 and entitled “Approximating Motion Capture. . . . Using a Single IMU Device” and in U.S. Ser. No. 18/939,288 entitled “Advanced Gait Analysis” and filed Nov. 6, 2024, of which the present application is a continuation-in-part, both co-owned, both hereby incorporated by reference in their entirety.
h. Increased cognitive load: as vision decreases, the person may need to pay more attention to auditory, tactile, and proprioceptive cues. This may lead to all or any subset of reduced dual-tasking ability during gait, fatigue, or slower movement execution.
i. Shortened stride length, e.g. to maintain better balance by reducing risk of misjudging obstacles or uneven terrain.
j. Swaying or shuffling: Some individuals with visual impairments may sway more or may shuffle more, e.g. feet are lifted less and instead slide along the floor, probably to minimize the chance of misjudging distances or tripping.

Training data may be generated by recording walks, then instructing the participants to shuffle, and recording their walks under this instruction.

Shuffling gait may be identifiable as IMU data characterized by small strides (e.g. trend analysis to identify that an end-user's stride length is getting shorter) and/or each stride being longer in duration; this may be detected, e.g. by measuring the time between each step/stride and determining whether there is a decrease in cadence (steps per minute).

Other gait parameters which may aid in identifying shuffling hence may be found, e.g. by trend analysis to change over time in persons who are deteriorating visually, may include:
Toe clearance: vertical distance between toes and ground during walking. Shuffling may result in reduced or no toe clearance, since the shuffling end-user's toes drag along the floor or barely lift off the ground; and/or
Ground reaction force (GRF): pressure exerted by the foot on the ground during walking. Lack of foot clearance due to shuffling may yield different patterns of pressure or force distribution, compared to normal walking which includes foot clearance; and/or
Knee and hip flexion: normal walking involves coordinated flexion of the knee and hip to lift the leg and move it forward, whereas in shuffling, decreased knee or hip flexion may occur.

It is appreciated that, typically, a single identification of shuffling (or of staring at the ground, as described elsewhere) does not result in an alert being generated by the system. Instead, suitable logic may be defined e.g. the system may conduct further passive monitoring of end-user Julia's gait, if shuffling is identified, and if three or more (say) walks which involve shuffling are detected within a 2-day (say) period, an alert may be generated by the system.
k. Head position: keep their head slightly down or tilt it forward, which may alter the natural flow of gait e.g. may cause the spine to lean forward, shifting the body's center of gravity, or may exhibit more side-to-side sway in the pelvis or hips, or may experience increased tension in the neck, shoulders, and upper back which may spread down to the lower body, causing a more rigid gait.

It is appreciated that the system may detect changes in the gait pattern which occur when a person begins to have an abnormal head position e.g. by machine learning patterns of people who stare at the floor, say by training a classifier using training data, which may include:
i. IMU data of persons walking, after these persons have been instructed to stare at the floor, and
ii. IMU data of persons walking who have not been so instructed.

The training data may, for example, include two IMU data sequences for each of 100 subjects, e.g. a 1st sequence where a subject walks normally from a to b, and a 2nd sequence where the same subject takes the same walk from point a to point b, this time after having been instructed to stare at the floor. A classifier may then be trained to distinguish end-users staring at the floor from end-users who are not.

It is appreciated that once the classifier is so trained, the classifier may be further improved using any known machine learning techniques such as but not limited to online/incremental/active/transfer learning, model ensembling, hyperparameter tuning, model pruning.

Inability to detect, or, alternatively, tendency to avoid obstacles known to the system like curbs, stairs, or changes in surface texture, or alternatively user may raise their feet higher to avoid tripping or exhibit hesitant or slow gait when approaching obstacles whose locations are known to the system. Training data may, for example, be generated by recording walks, then instructing the end-users who did the walking to raise their feet higher, and recording their walks under that instruction.

Any suitable gait analysis technique may be employed to detect that a person has begun to keep their head slightly down or tilt it forward (to better use remaining vision and/or to listen for auditory cues that help with navigation, typically depending on how the person's new head position alters the their gait, e.g. all or any subset of:
1. Altered Posture and Balance, which may shift the body's center of gravity slightly, leading to a more forward-leaning posture, or leaning more on their lower body or different distribution of their weight while walking. The methods described in co-owned U.S. Ser. No. 18/939,288 entitled SYSTEM, METHOD AND COMPUTER PROGRAM PRODUCT FOR ADVANCED GAIT ANALYSIS, from which continuation-in-part status is claimed herein and which is hereby incorporated by reference in its entirety, may be used to detect such situations, e.g. by detecting the structure of the trajectory of the center of pressure over the gait cycle, using suitable kinetic gait measures such as a graph of center of pressure and/or its parameters.
2. Tilting the head forward to stare at the ground may cause a slight shortening of the stride (decrease in stride-length e.g.) which may be extracted from IMU data via gait analysis.

3. Increasing muscle tension in the neck and upper body, leading to a stiffer gait which may be detected automatically, e.g.by identifying decreased range of motion in the lower limbs and trunk, e.g. a person who does not bend their knees or hips fully while walking, and instead employs straight-legged and/or jerky movements; lack of smooth transition between steps may be detected by analyzing the pattern of footfall.

The system herein, which does not rely on in-clinic evaluation of visual decline, is particularly advantageous because frail persons, disabled persons, elders, persons with chronic health conditions, and demented persons, tend to suffer from visual problems at a higher rate than the general population. For example, macular degeneration, cataracts, diabetic retinopathy, and glaucoma are more common in older adults. Also, a frail person may have diminished ability to recover from visual impairment and indeed all health issues. Also, some disabilities may be linked to a higher incidence of visual impairments, e.g. cerebral palsy, autism, or Down syndrome may increase the risk for refractive errors, strabismus, nystagmus, or cataracts.

Some disabled individuals may be undergoing regimes or treatments that increase the risk of eye problems (e.g., corticosteroids may lead to cataract development).

Physical changes in the brain that cause dementia may also impact the visual processing centers in the brain, leading to increased difficulty with vision.

Dementia increases the risk of other age-related conditions that affect vision, such as cataracts, macular degeneration, or diabetic retinopathy.

All of these groups may experience difficulties in facilitating or cooperating with active, in-clinic visual monitoring. This may also be true of persons with low socioeconomic status who may also live in environments that contribute to eye strain or injury and/or may experience disparities in healthcare access, and minority populations who may have a higher predisposition to certain eye conditions, e.g. African Americans are at a higher risk for glaucoma, and Hispanics and Latinos are at risk for diabetic retinopathy.

Another advantage of embodiments herein is that the health system is challenged to provide visual health monitoring to frail, disabled, demented individuals, as well as other vulnerable populations, such as older adults, people with chronic health conditions, and those with limited access to healthcare. This is due to physical barriers such as limited mobility or transportation difficulties, existence of underserved areas, challenges inherent in telemedicine for eye care which has failed to gain traction in providing remote consultations, vision assessments, and teleophthalmology for persons who cannot visit a clinic easily.

Another advantage is that populations at higher risk of visual problems, such as people with diabetes or hypertension, may be the recipients of routine eye screenings, however, in practice this is often not the case, even when health insurance providers, including Medicare, cover the cost of these screenings.

The system herein is easily adoptable since no new deployment is required; persons having cellphones may easily participate without further ado, as opposed to attempts to introduce vision screening in schools, nursing homes and senior centers, and home visits for demented or frail individuals, where home health nurses may monitor vision and so forth. Note, for example, that a demented person may not remember to go for eye exams, or may become confused or anxious during a clinic visit, whereas the embodiments herein may be administered entirely passively and/or without the cooperation of the examined subject, which is also advantageous for persons who have language barriers vis-a-vis their caregiver population. Finally, conventional vision care is often inaccessible for anyone lacking insurance coverage. However, these same persons may well bear a cellphone, facilitating the administration of vision monitoring for low-income persons.

It is appreciated that monitoring according to embodiments herein may improve the quality of life for persons with currently undetected visual impairments, e.g. if responsive to gait deterioration detected as described herein, the system automatically dispatches suitable state-of-the-art wearable technologies, such as smart glasses or head-mounted cameras, which aid visually impaired individuals to better navigate their environments.

It is appreciated that the system may, e.g. if a standard visual impairment outcome is found to have worsened or found to be below-threshold, or if a heuristic such as shuffling or staring at the ground is detected, prompt an end-user to undertake tests via her or his mobile phone. It is appreciated that various relevant tests (e.g. for testing peripheral vision) are known which may be undertaken without an in-clinic visit, e.g. as evidenced by mobile apps which monitor and record changes in vision, sending updates to healthcare providers for ongoing care. These mobile apps include:

Amsler Grid (by Eye Health) used to monitor macular degeneration (AMD) and other vision changes. The app provides a digital version of the Amsler grid to help users detect visual distortions such as wavy lines, blurred areas, or blank spots that may indicate macular degeneration or other retinal problems.

Glaucoma App helps users monitor their vision for signs of glaucoma, such as peripheral vision loss. MyVisionTrack (by 20/20), designed for people with diabetic retinopathy, age-related macular degeneration (AMD), or diabetic macular edema (DME) provides a series of simple vision tests that track any changes in vision, for example:

Regular vision tests using customizable grids and patterns.

VisionTest (by ORA), which includes tests for visual acuity, color vision, and contrast sensitivity.

Diabetic Eye Screening, which helps individuals with diabetes monitor their eye health, specifically for signs of diabetic retinopathy by providing visual field tests, inter alia.

Vivid Vision, which provides vision therapy exercises for improving depth perception, eye coordination, and other visual functions and tracks an end-user's performance.

It is appreciated that the same frail, disabled, elderly, and demented populations who are less likely to be monitored for vision, are also more seriously impacted by the visual problems they are not being monitored for, compared to others with the same visual issues, but do not belong to these vulnerable groups. For example, frail individuals are more seriously impacted because their diminished physical strength and stamina make it difficult to adapt to changes required by a new and/or progressing visual impairment.

Also, vision impairment is a significant risk factor for falls. At-risk persons, whose fall risk is already high, may be at even higher risk if they suffer a visual impairment that further raises this risk, leading to fractures which again further reduce their mobility and quality of life. Disabled individuals may be more seriously impacted, e.g. if they have other health conditions that exacerbate visual problems. For example, cerebral palsy may be associated with both motor impairments and visual issues. Dementia, particularly Alzheimer's disease, may be unable to recognize hazards like stairs or objects in their path, making them more vulnerable to accidents than other visually impaired persons who have, at least, a better ability to recognize whatever obstacles they may manage to see.

Finally, recovery from cataract surgery may be slower, or rehabilitation to address balance issues related to vision loss may be less effective, for challenged populations.

It is appreciated that an end-user's vision helps the end-user maintain his or her balance, inter alia. Therefore, parameters typical of wobbly gait may also be used as indicators of visual deterioration, such as but not limited to all or any subset of the following: increased step width, relative to a person's own norms or relative to population, e.g. as end-user tries to improve balance by increasing her or his base of support by widening their stance to avoid falling; decreased stride length, increased cadence, reduced foot clearance, reduced trunk stability, e.g. swaying or instability in the upper body during walking, such as side-to-side or forward-backward swaying while standing or walking which is excessive relative to the person's own norms or relative to population norms, increased double support time, slower gait speed, altered foot progression angle, e.g. more out-toeing or in-toeing gait to compensate for instability, reduced arm swing (which may be measurable depending on the bodily position of the phone which is typically known to the system, e.g. as described in co-owned US patent Publication 20200289027 incorporated herein by reference in its entirety which describes, inter alia, how to configure a system to monitor gait of an end-user bearing a wearable device, e.g. cellphone equipped with at least one magneto-inertial sensor or IMU, the system comprising a processor configured to receive raw sensor data from the wearable device's at least one magneto-inertial sensor and to extract situational data from the raw sensor data, which includes the cellphone's bodily position relative to the end-user.

The term “gait analysis” as used herein is intended to include derivation of any known gait parameters from IMU data, including but not limited to gait parameters specifically mentioned herewithin.

According to an embodiment, the system of FIG. 1 may be used to predict high blood pressure e.g. regarding high blood pressure as a standard outcome and using the method of FIG. 2, applied to high blood pressure rather than to IOP, typically assuming that some end-users in the system have blood-pressure readings stored in the system, and that some of these end-users suffer from hypertension, whereas others do not.

It is appreciated that one reason for conducting routine eye exams is that these may pick up on undiagnosed hypertension e.g. due to the damage that such hypertension may cause to the retina's blood vessels. However, according to certain embodiments, the system herein may provide or augment the hypertension screening functionality of routine eye exams in the clinic, given that hypertension may give rise to “indicator” gait parameter values or patterns which differ from the gait parameter values or patterns (e.g. over time) of users who do not suffer from hypertension hence suggestion presence of hypertension. For example, high blood pressure (which fluctuates e.g.) may adversely affect a person's balance and/or gait, resulting, say, in shuffling, wobbliness, limping, difficulty walking for long distances or slow gait, and/or may generate muscle stiffness due to poor circulation over time and/or may may reduce joint flexibility, resulting in less smooth gait. Also, brain dysfunction due to untreated blood pressure which becomes extremely high may impair motor control e.g. causing unsteady walking or shuffling. An undiagnosed stroke caused by (typically uncontrolled) high blood pressure may also change gait parameters. Thus the system may for example generate a suggestion that a given end-user (e.g. who has been found to be shuffling, or for whom hypertension has been estimated to be present using the method of FIG. 2) should undergo a simple blood pressure test. If the outcome of the test (the user's measured blood pressure) is entered in the system (e.g. if such a data entry is system-required to enable a clinician to “close the alert”), the system may further improve e.g. by training a model to predict blood pressure, based on training data which pairs time-stamped gait parameters in the system with blood pressure “labels” of the same user and having the same or similar time-stamp, selecting a training group of users some of whom do have hypertension and others of whom do not.

It is appreciated that detection of tripping episodes according to any embodiment herein may have various advantages. For example, each tripping episode is associated with a time-stamp T and with a cellphone-bearing end-user who tripped, whose location at time T (e.g. via GPS) is known. Thus the system herein may alert local authorities of locations which are causing tripping and potentially, causing falls, for maintenance purposes. Alternatively or in addition, circumstances under which end-users as a whole or individuals are likely to trip (e.g. a poorly maintained sidewalk or even black ice in a given location, or certain combinations of variables e.g. times of day, age, states of health) may be identified by the system e.g. in order to generate warnings to end-users to beware of tripping each time such circumstances are present. Such users may then be reminded to walk more slowly, to watch the ground, to use their phone flashlight, or simply to avoid walking at all under the current circumstances, all in order to improve the current appalling fall statistics e.g. according to WHO, an estimated 684000 fatal falls occur annually, making falls the second leading cause of unintentional injury death.

Herein, “passive” is intended to include IMU data collection from a user's phone's IMU, which does not require user cooperation each time the IMU data is collected and in fact the user may be unaware that IMU data is being collected e.g. because IMU data collection and subsequent analysis do not require that the user be prompted to provide any information or to perform any particular action. For example, the system typically does not require a user to be prompted to walk or to indicate whether or not s/he is walking. Instead, the disclosures of co-owned U.S. Ser. No. 18/975,890 entitled “Advanced Pedestrian Navigation Based on Inertial Gait Analysis and GPS Data” and/or of co-owned US20220111257 entitled “System . . . for Sensor-Based Enhancement of Physical Rehabilitation” (both of which are hereby incorporated herein by reference in their entirety) may be used to automatically classify a given time-interval as including gait or not e.g. by determining whether the user is ambulating during that interval. Then, IMU data may if desired to be collected only during such intervals which, as automatically determined, do include ambulation as opposed to intervals in which an end-user is stationary e.g.

It is appreciated that conventionally, annual eye exams in-clinic are advantageous:
a. to maintain clear vision thereby to maintain quality of life including by avoiding injuries such as falls, by identifying new or worsening eye conditions such as but not limited to cataracts, macular degeneration, glaucoma, diabetic retinopathy, reduced field of view, reduced night vision, or myopia, and/or
b. because an eye exam is a relatively easy way to screen, in a single session, for a variety of new health issues such as but not limited to the following diseases: high blood pressure (hypertension), diabetes and pre-diabetes, brain tumors, high cholesterol (hyperlipidemia), multiple sclerosis, thyroid disease (hypothyroidism or hyperthyroidism), autoimmune diseases such as rheumatoid arthritis, lupus, or sarcoidosis, certain cancers such as leukemia or lymphoma, chronic kidney disease, sleep apnea and HIV/AIDS.

According to certain embodiments, the system herein may be used to prioritize eye-exams by identifying, based on gait analysis, those cellphone bearing end-users whose visual health may have newly deteriorated.

It is appreciated that the system herein may improve as it matures. For example, if the system does recommend an eye-exam for certain flagged end-users by issuing alerts for each of these, the medical organization may give back feedback for each user so flagged e.g. was an issue found that justified the eye-exam, or was no issue found. Typically, each time a certain amount of feedback accumulates (e.g. each time the amount of newly accumulated feedback exceeds, say, 5% or 10% or 15% of the amount of previously accumulated feedback) the system learns from the feedback e.g. by adjusting the alert thresholds, on occasion or dynamically in real time, to achieve false negative and false positive rates defined by a given HMO as desirable. It is appreciated also, that the system may initially use thresholds defined by validated standard outcomes known in the art e.g. for IOP, which may be applied to estimated standard outcomes derived from gait parameters as per any embodiment herein or any embodiment described in co-owned patent documents incorporated herein by reference, resulting in an immediately useful system. As feedback accumulates however, and is used as labels, the system may learn thresholds for the gait parameters themselves, which may then be applied to the gait parameters themselves instead of or in addition to applying thresholds defined by validated standard outcomes to system-generated standard outcome estimates.

Alternatively or in addition, a mature system may include, in at least one system-generated alert, a specific condition to be confirmed/ruled out when the user arrives at the clinic e.g. “brain tumor” or “kidney disease” or other diseases which impact gait in various ways, e.g brain tumors and multiple sclerosis may directly affect a user's brain's motor control, diabetes and chronic kidney disease may damage nerves or weaken muscles, etc. The system may learn this if the feedback from the HMO indicates which alerts resulted in certain diseases being confirmed (by in-clinic testing and/or examination) and which resulted in the same diseases being ruled out. Since gait deterioration patterns of these different diseases may be found to be different, labels from eye doctors (which can be confirmed or not by other clinicians and testing to which the eye doctors refer) may be used as dependent variables, thus the system may suggest which diagnoses to consider using gait parameters and/or trends thereof over days, weeks, months and years, as independent variables.

Any label may be used for training the system herein to further improve as it works, e.g. any suitable measure of any suitable clinical outcome. It is appreciated that the system herein may serve plural HMOs and each HMO may train the system to improve using its own desired clinical outcomes as labels.

It is appreciated that terminology such as “mandatory”, “required”, “need” and “must” refer to implementation choices made within the context of a particular implementation or application described herewithin for clarity, and are not intended to be limiting, since, in an alternative implementation, the same elements may be defined as not mandatory and not required. or may even be eliminated altogether.

Components described herein as software may, alternatively, be implemented wholly or partly in hardware and/or firmware, if desired, using conventional techniques, and vice-versa. Each module or component or processor may be centralized in a single physical location or physical device or distributed over several physical locations or physical devices.

Included in the scope of the present disclosure, inter alia, are electromagnetic signals in accordance with the description herein. These may carry computer-readable instructions for performing any or all of the operations of any of the methods shown and described herein, in any suitable order, including simultaneous performance of suitable groups of operations, as appropriate. Included in the scope of the present disclosure, inter alia, are machine-readable instructions for performing any or all of the operations of any of the methods shown and described herein, in any suitable order; program storage devices readable by machine, tangibly embodying a program of instructions executable by the machine to perform any or all of the operations of any of the methods shown and described herein, in any suitable order, i.e., not necessarily as shown, including performing various operations in parallel or concurrently, rather than sequentially, as shown; a computer program product comprising a computer useable medium having computer readable program code, such as executable code, having embodied therein, and/or including computer readable program code for performing, any or all of the operations of any of the methods shown and described herein, in any suitable order; any technical effects brought about by any or all of the operations of any of the methods shown and described herein, when performed in any suitable order; any suitable apparatus or device or combination of such, programmed to perform, alone or in combination, any or all of the operations of any of the methods shown and described herein, in any suitable order; electronic devices each including at least one processor and/or cooperating input device and/or output device and operative to perform, e.g., in software, any operations shown and described herein; information storage devices or physical records, such as disks or hard drives, causing at least one computer or other device to be configured so as to carry out any or all of the operations of any of the methods shown and described herein, in any suitable order; at least one program pre-stored e.g. in memory or on an information network such as the Internet, before or after being downloaded, which embodies any or all of the operations of any of the methods shown and described herein, in any suitable order, and the method of uploading or downloading such, and a system including server/s and/or client/s for using such; at least one processor configured to perform any combination of the described operations or to execute any combination of the described modules; and hardware which performs any or all of the operations of any of the methods shown and described herein, in any suitable order, either alone or in conjunction with software. Any computer-readable or machine-readable media described herein is intended to include non-transitory computer- or machine-readable media.

Any computations or other forms of analysis described herein may be performed by a suitable computerized method. Any operation or functionality described herein may be wholly or partially computer-implemented, e.g., by one or more processors. The invention shown and described herein may include (a) using a computerized method to identify a solution to any of the problems or for any of the objectives described herein, the solution optionally including at least one of a decision, an action, a product, a service or any other information described herein that impacts, in a positive manner, a problem or objectives described herein; and (b) outputting the solution.

The system may, if desired, be implemented as a network—e.g., web-based system employing software, computers, routers, and telecommunications equipment, as appropriate.

Any suitable deployment may be employed to provide functionalities, e.g., software functionalities shown and described herein. For example, a server may store certain applications, for download to clients, which are executed at the client side, the server side serving only as a storehouse. Any or all functionalities, e.g., software functionalities shown and described herein, may be deployed in a cloud environment. Clients, e.g., mobile communication devices such as smartphones, may be operatively associated with, but external to the cloud.

The scope of the present invention is not limited to structures and functions specifically described herein and is also intended to include devices which have the capacity to yield a structure, or perform a function, described herein, such that even though users of the device may not use the capacity, they are, if they so desire, able to modify the device to obtain the structure or function.

Any “if-then” logic described herein is intended to include embodiments in which a processor is programmed to repeatedly determine whether condition x, which is sometimes true and sometimes false, is currently true or false, and to perform y each time x is determined to be true, thereby to yield a processor which performs y at least once, typically on an “if and only if” basis, e.g., triggered only by determinations that x is true, and never by determinations that x is false.

Any determination of a state or condition described herein, and/or other data generated herein, may be harnessed for any suitable technical effect. For example, the determination may be transmitted or fed to any suitable hardware, firmware, or software module, which is known or which is described herein to have capabilities to perform a technical operation responsive to the state or condition. The technical operation may, for example, comprise changing the state or condition, or may more generally cause any outcome which is technically advantageous, given the state or condition or data, and/or may prevent at least one outcome which is disadvantageous, given the state or condition or data. Alternatively, or in addition, an alert may be provided to an appropriate human operator or to an appropriate external system.

Features of the present invention, including operations which are described in the context of separate embodiments, may also be provided in combination in a single embodiment. For example, a system embodiment is intended to include a corresponding process embodiment, and vice versa. Also, each system embodiment is intended to include a server-centered “view” or client centered “view”, or “view” from any other node of the system, of the entire functionality of the system, computer-readable medium, apparatus, including only those functionalities performed at that server or client or node. Features may also be combined with features known in the art, and particularly, although not limited to those described in the Background section or in publications mentioned therein.

Conversely, features of the invention, including operations, which are described for brevity in the context of a single embodiment or in a certain order, may be provided separately or in any suitable sub-combination, including with features known in the art (particularly although not limited to those described in the Background section or in publications mentioned therein) or in a different order. “e.g.” is used herein in the sense of a specific example which is not intended to be limiting. Each method may comprise all or any subset of the operations illustrated or described, suitably ordered e.g. as illustrated or described herein.

Devices, apparatus or systems shown coupled in any of the drawings may in fact be integrated into a single platform in certain embodiments, or may be coupled via any appropriate wired or wireless coupling, such as but not limited to optical fiber, Ethernet, Wireless LAN, HomePNA, power line communication, cell phone, Smart Phone (e.g. iPhone), Tablet, Laptop, PDA, Blackberry GPRS, Satellite including GPS, or other mobile delivery. It is appreciated that in the description and drawings shown and described herein, functionalities described or illustrated as systems and sub-units thereof may also be provided as methods and operations therewithin, and functionalities described or illustrated as methods and operations therewithin may also be provided as systems and sub-units thereof. The scale used to illustrate various elements in the drawings is merely exemplary and/or appropriate for clarity of presentation, and is not intended to be limiting.

Any suitable communication may be employed between separate units herein, e.g., wired data communication and/or in short-range radio communication with sensors such as cameras e.g., via Wifi, Bluetooth, or Zigbee.

It is appreciated that implementation via a cellular app as described herein is but an example, and, instead, embodiments of the present invention may be implemented, say, as a smartphone SDK; as a hardware component; as an STK application, or as suitable combinations of any of the above.

Any processing functionality illustrated (or described herein) may be executed by any device having a processor, such as but not limited to a mobile telephone, set-top-box, TV, remote desktop computer, game console, tablet, mobile e.g. laptop or other computer terminal, embedded remote unit, which may either be networked itself (may itself be a node in a conventional communication network e.g.) or may be conventionally tethered to a networked device (to a device which is a node in a conventional communication network, or is tethered directly or indirectly/ultimately to such a node).

Any operation or characteristic described herein may be performed by another actor outside the scope of the patent application and the description is intended to include apparatus whether hardware, firmware or software which is configured to perform, enable, or facilitate that operation or to enable, facilitate, or provide that characteristic.

The terms processor or controller or module or logic as used herein are intended to include hardware such as computer microprocessors or hardware processors, which typically have digital memory and processing capacity, such as those available from, say Intel and Advanced Micro Devices (AMD). Any operation or functionality or computation or logic described herein may be implemented entirely or in any part on any suitable circuitry including any such computer microprocessor/s as well as in firmware or in hardware or any combination thereof.

It is appreciated that elements illustrated in more than one drawing, and/or elements in the written description, may still be combined into a single embodiment, except if otherwise specifically clarified herewithin. Any of the systems shown and described herein may be used to implement or may be combined with, any of the operations or methods shown and described herein.

It is appreciated that any features, properties, logic, modules, blocks, operations, or functionalities described herein which are, for clarity, described in the context of separate embodiments, may also be provided in combination in a single embodiment, except where the specification or general knowledge specifically indicates that certain teachings are mutually contradictory and cannot be combined. Any of the systems shown and described herein may be used to implement or may be combined with, any of the operations or methods shown and described herein.

Conversely, any modules, blocks, operations or functionalities described herein, which are, for brevity, described in the context of a single embodiment, may also be provided separately or in any suitable sub-combination, including with features known in the art. Each element, e.g., operation described herein may have all characteristics and attributes described or illustrated herein, or, according to other embodiments, may have any subset of the characteristics or attributes described herein.

It is appreciated that apps implementing any functionality herein may include a cell app, mobile app, computer app, or any other application software. Any application may be bundled with a computer and its system software, or published separately. The term “phone” and similar used herein is not intended to be limiting and may be replaced or augmented by any device having a processor, such as but not limited to a mobile telephone, or also set-top-box, TV, remote desktop computer, game console, tablet, mobile, e.g., laptop or other computer terminal, embedded remote unit, which may either be networked itself (may itself be a node in a conventional communication network e.g.) or may be conventionally tethered to a networked device (to a device which is a node in a conventional communication network or is tethered directly or indirectly/ultimately to such a node). Thus, the computing device may even be disconnected from e.g., Wifi, Bluetooth, etc., but may be tethered directly or ultimately to a networked device.

References herein to “said (or the) element x” having certain (e.g., functional or relational) limitations/characteristics, are not intended to imply that a single instance of element x is necessarily characterized by all the limitations/characteristics. Instead, “said (or the) element x” having certain (e.g. functional or relational) limitations/characteristics is intended to include both (a) an embodiment in which a single instance of element x is characterized by all of the limitations/characteristics and (b) embodiments in which plural instances of element x are provided, and each of the limitations/characteristics is satisfied by at least one instance of element x, but no single instance of element x satisfies all limitations/characteristics. For example, each time L limitations/characteristics are ascribed to “said” or “the” element X in the specification or claims (e.g. to “said processor” or “the processor”), this is intended to include an embodiment in which L instances of element X are provided, which respectively satisfy the L limitations/characteristics, each of the L instances of element X satisfying an individual one of the L limitations/characteristics. The plural instances of element x need not be identical. For example, if element x is a hardware processor, there may be different instances of x, each programmed for different functions and/or having different hardware configurations (e.g., there may be 3 instances of x: two Intel processors of different models, and one AMD processor).

1. An improved health-care system comprising:
an output indication generating functionality configured to screen for visual health deterioration by generating an output indication aka alert to at least one entity to alert for visual health deterioration; and
a visual health monitoring functionality including a hardware processor configured to monitor persons carrying cellphones for at least one gait impairment indicator which may be characteristic of deterioration of visual health, by performing gait analysis, at intervals, on data generated by the persons' cellphones' IMUs, and, accordingly, to command the output indication generating functionality to generate the output indication e.g. when at least one gait impairment indicator monitored complies with at least one alert criterion.

2. The system of claim 1, wherein the alert stipulates at least one change in gait parameter/s relative to value/s of said gait parameter/s that has/have characterized an individual user in the past.

3. The system of claim 1, wherein the alert stipulates at least one estimated standard outcome which deviates from at least one norm, stored in the system, for the standard outcome.

4. The system of claim 1, wherein the visual impairment characteristic of deterioration of visual health comprises plural changes in plural gait parameters respectively which are machine-learned to be indicative of deterioration of visual health.

5. The system of claim 3, wherein said estimated standard outcome, is generated using a model trained on training data including IMU data recorded for end-users labelled to indicate standard outcome results for each end-user which were generated in-clinic.

6. The system of claim 1, wherein responsive to the alert, the system may schedule an in-clinic session with a digital retinal camera.

7. The system of claim 1, wherein responsive to the alert, the system may schedule an in-clinic optical coherence tomography (OCT) session.

8. The system of claim 3, wherein said estimated standard outcome, is generated using a classifier trained on training data including first IMU data recorded for first end-users labelled to indicate that, for these end-users, a given standard outcome has deteriorated, and second IMU data recorded for second end-users which is labelled to indicate that, for these end-users, the given standard outcome has not deteriorated.

9. The system of claim 1, wherein said gait impairment characteristic of deterioration of visual health comprises an indication of shuffling generated by gait analysis.

10. The system of claim 1, wherein said gait impairment characteristic of deterioration of visual health comprises an indication generated by gait analysis that an end-user is staring at the ground when walking.

11. The system of claim 1, and also comprising appointment scheduling functionality configured to prompt a user and/or his eye physician, responsive to said output indication, to schedule an appointment in-clinic to undergo eye exams e.g. all or any subset of visual acuity tests: tonometry and dilated eye exams to identify at least one of macular degeneration, diabetic retinopathy, or retinal detachment.

12. The system of claim 1, wherein upon detection of gait impairment characteristic of deterioration of visual health, the system is configured to prompt undergo remote monitoring e.g. apps e.g. for Amsler grid and/or initiate home testing e.g. to undergo a home tonometer test to measure intraocular pressure from home.

13. The system of claim 1, wherein said at least one visual health deterioration indicator comprises an indication that a person has begun shuffling, and wherein the hardware processor comprises a classifier configured to differentiate persons who shuffle from persons who do not shuffle.

14. The system of claim 1, wherein said at least one visual health deterioration indicator comprises an indication that a person has begun staring at the ground during gait and wherein the hardware processor comprises a classifier configured to differentiate persons who stare at the ground during gait from persons who do not.

15. An improved health-care method comprising:
Using an output indication generating functionality to screen for visual health deterioration by generating an output indication aka alert to at least one entity to alert for visual health deterioration; and
Using a hardware processor to monitor persons carrying cellphones for gait impairment characteristic of deterioration of visual health by repeatedly performing gait analysis on data generated by the persons' cellphones' IMUs, and, accordingly, commanding the output indication generating functionality to generate the output indication.

16. A computer program product, comprising a non-transitory tangible computer readable medium having computer readable program code embodied therein, said computer readable program code adapted to be executed to implement an improved health-care method comprising:
Using an output indication generating functionality to screen for visual health deterioration by generating an output indication aka alert to at least one entity to alert for visual health deterioration; and
Using a hardware processor to monitor persons carrying cellphones for gait impairment characteristic of deterioration of visual health by repeatedly performing gait analysis on data generated by the persons' cellphones' IMUs, and, accordingly, commanding the output indication generating functionality to generate the output indication.
